(19) United States
(12) Patent Application Publication
(10) Pub. No.: US 2005/0176068 A1
(43) Pub. Date: Aug. 11, 2005

(54) Direct cell target analysis

(76) Inventors: Michael R. Emmert-Buck, Silver Springs, MD (US); Mamoun Ahram, Kennewick, WA (US)

Correspondence Address: KLARQUIST SPARKMAN, LLP, 121 S.W. SALMON STREET, SUITE #1600, ONE WORLD TRADE CENTER, PORTLAND, OR, 97204-2988, US

(21) Appl. No.: 10/511,511

(22) PCT Filed: Apr. 25, 2003
(86) PCT No.: PCT/US03/12734

Related U.S. Application Data
(60) Provisional application No. 60/375,727, filed on Apr. 26, 2002.

(51) Int. Cl. C12Q 1/68; G01N 33/53; G01N 33/567

(52) U.S. Cl. 435/7.2; 435/6

(57) ABSTRACT

Disclosed herein are Direct Cell Target Analysis (“DCTA”) molecules and Direct Cell Target (“DCT”) methods for directly targeting and acting upon biomolecules. These methods and molecules can be used with specific cells within complex, heterogeneous tissue such that target biomolecules can be procured for subsequent analysis or directly analyzed without the need for physical separation of the biomolecules from other cells or cell components in the population. In general, the methods involve use of a fusion molecule having a first moiety to identify and localize target cells within a tissue sample, and a second moiety to generate detectable products within the target cells that may be detected and subsequently analyzed, and optionally isolated.

### FIELD

The present disclosure is related to methods of analysis of biomolecules. Particular methods involve targeting and acting upon biomolecules in specific target cells in a heterogeneous population for subsequent analysis and/or separation of components of interest from their surroundings.

### BACKGROUND

Tissue microdissection includes a broad category of techniques used to harvest specific cells or cell populations from a histological sample under direct microscopic visualization. Original microdissection techniques involved painstaking (and sometimes clumsy) manual dissection using needles or other micro-manipulation devices to isolate individual cells based on visible, histological characteristics. Recent advances have led to more precise microdissection techniques, one of which is laser-capture microdissection (LCM) (see, for instance, Gillespie et al, Cancer J. 7(1): 32-39, January/February 2001, U.S. Pat. No. 5,843,657; and publication WO 00/49410 (International Patent Application No. PCT/US00/04023)).

LCM involves placing a transparent, thermoplastic film on top of a thin tissue section and activating the film directly over the cell(s) of interest using a pulse from a focused electromagnetic energy source (e.g., laser beam). The laser melts the film onto the top of the tissue sample, thereby adhering the film to the cell(s) of interest. When peeled away from the tissue sample, the film carries the fused cells (and their contents) along. Constituents, such as nucleic acids or proteins, can then be extracted from the dissected cells and used for molecular profiling. Using LCM, an operator can manually procure cells on the order of hundreds of cells per hour. Though LCM is a much more efficient and precise technique than manual microdissection, the process of identifying cells of interest through the microscope, and pulsing each individual cell with the laser, is tedious and not entirely amenable to automation.

Other microdissection techniques involve overlaying a photoresist (such as those used in etching computer chips) onto a thin tissue section, then activating specific regions of the photoresist using electromagnetic radiation (e.g., a beam of a laser). Depending on the photoresist used, the “desired” cells are either washed off in the activated areas, or the undesired cells are washed away while the activated photoresist holds the desired cells to the slide. These methods share the same inherent disadvantages of LCM, in that individual cells must be visually identified and targeted before harvest.

Methods to directly analyze protein or nucleic acid (e.g., DNA or mRNA) in specific cells in a heterogeneous population, without the need to physically separate and procure cells, have previously been unavailable. Additionally, methods of automatically selectively identifying cells for subsequent isolation by microdissection have been unavailable. Thus, improved methods of direct molecular analysis of cells without a separate microdissection step and/or isolation of cells of interest are needed, particularly methods that have the potential of being automated.

### SUMMARY OF THE DISCLOSURE

Described herein are Direct Cell Target Analysis (“DCTA”) molecules and Direct Cell Target (“DCT”) methods for directly targeting and acting upon biomolecules. These methods and molecules can be used to facilitate interaction with specific cells within complex, heterogeneous tissue such that the target biomolecules can be procured for subsequent analysis or directly analyzed without the need for physical separation of the biomolecules from cells or cell components in the population.

Specific Direct Cell Target analysis methods involve the use of DCTA molecules to facilitate targeted analysis. DCTA molecules contain at least two functional moieties, the combination of which allows a user to analyze biomolecules based upon the distribution of the biomolecules within a sample. DCTA molecules are used to target specific cells (or components within cells) of interest using a localizing or “targeting” moiety, and act upon those cells, or components of those cells, using a functional or “active” moiety that generates a detectable signal, thereby facilitating the targeted analysis.

In certain methods of targeted analysis, the cells or components of interest are physically removed from their environment. In others, cells or components are simply distinguished from their environment such that they can be visualized or characterized without removing them from that environment. Methods provided herein obviate the need for mechanical microdissection, and allow the user to proceed directly to subsequent procedures, such as molecular analysis of targeted components or cells.

Contemplated DCTA molecule embodiments include (but are not limited to): (1) antibody-reverse transcriptase fusion proteins or molecules, whereby cells containing such a DCTA molecule support in situ transcription following activation; (2) antibody-DNA polymerase fusion proteins or molecules, whereby cells containing such a DCTA molecule support in situ polymerization following activation; and (3) antibody-lactoperoxidase fusion proteins or molecules, whereby tyrosine and tryptophan residues within a target cell containing such a DCTA molecule are labeled by activation of the lactoperoxidase.

Examples of provided DCTA molecules include those whereby target cell proteins are tagged for subsequent identification by incorporation of a labeled component (e.g., a radioactively labeled compound (e.g., a nucleotide), fluorophore, chromophore, heavy or light isotope labeled affinity tag (ICAT) reagents with (heavy) or without (light) deuterium, etc.), and DCTA molecules wherein the targeting moiety is a DNA probe fused to an active protein moiety.

The present disclosure further relates to various applications of the methods disclosed and molecules. Specific embodiments permit non-mechanical targeting of cells and cell components, by facilitating interaction of DCTA molecules with the tissue sample to be analyzed.

In a first specific embodiment there is provided a method of analyzing a tissue sample, which method involves contacting a Direct Cell Target Analysis (DCTA) molecule with the tissue sample under conditions that allow at least a portion of the DCTA molecule to interact with at least a portion of the tissue sample, wherein the DCTA molecule comprises a targeting moiety, capable of localizing the DCTA to target cells or components within the sample; and an active moiety, capable of generating a detectable signal or product; activating the active moiety of the DCTA molecule; and detecting the signal or product generated by the activated second moiety, thereby analyzing the tissue sample. Optionally, the DCTA molecule further comprises a linker, for instance a polymer linker joining the active moiety to the targeting moiety.

In specific examples, the tissue sample includes biopsy material, a tissue section, a cell culture preparation, a cytology preparation, cells in vitro, or cells in vivo. Optionally, one or more components that are necessary for generation of the detectable products are externally provided. For instance, a labeled nucleotide is provided in some embodiments, such as a nucleotide labeled with an isotope or a fluorophore.

By way of example, the targeting moiety used in described methods includes a variable region of an antibody binding domain, or more specifically it is in some embodiments a generalized targeting moiety and comprises a variable region of a secondary antibody binding domain. Alternatively, in other embodiments the targeting moiety includes a ligand that specifically binds to a receptor protein within or upon target cells in the tissue sample, or a nucleic acid molecule capable of hybridizing to a complementary sequence within the target tissue.

By way of example, the active moiety used in described methods includes a reverse transcriptase molecule and the detectable products are cDNA transcripts. In other examples, the active moiety includes a DNA polymerase molecule and the detectable products are DNA transcripts. In still other examples, the active moiety includes a lactoperoxidase molecule and the detectable products comprise iodinated tryptophan or tyrosine residues. The active moiety in some examples comprises lactoperoxidase and the detectable products comprise ¹²⁵I labeled proteins.

Also described are methods of analyzing a tissue sample using DCTA, wherein the detectable signal is visualized without physical separation of the analyzed products from the sample. Other example methods involve separating the detectable products from the sample prior to analysis. In yet other, possibly overlapping, examples, the method further involves quantifying the detectable products. Optionally, in many of the described methods the detectable products can be amplified during analysis.

In particular representative examples, the DCTA molecule comprises a poly(1-lysine hydrobromide) polymer conjugated to lactoperoxidase and goat anti-mouse IgG antibody.

In addition, other embodiments are methods for screening for a disease in a subject, which methods involve using a DCTA as described herein.

Further embodiments are kits for analysis of a sample, which kits include at least one DCTA molecule. Examples of such kits contain at least one standardized DCTA molecule. Other examples of described kits include at least one DCTA molecule that includes a targeting moiety specific for a disease-linked molecule (e.g., a disease-specific protein or a disease-specific nucleic acid molecule). Yet other example kits are for detection of a mutation in a sample, and include a DCTA molecule capable of targeting cells of interest in the sample and producing a detectable signal, whereby the signal provides information regarding whether the mutation is present. Still other kits are for determining whether a subject has a disease, and include a DCTA molecule capable of targeting cells of interest in the sample and producing a detectable signal, whereby the signal provides information regarding whether the subject has the disease.

Also provided herein are DCTA molecules for use in the described methods and kits.

The foregoing and other features and advantages will become more apparent from the following detailed description of several embodiments, which proceeds with reference to the accompanying figures.

### BRIEF DESCRIPTION OF THE FIGURES

FIG. 1A is a schematic representation of one Direct Cell Target Analysis (DCTA) molecule embodiment, illustrating a polymer, with an active moiety and a targeting moiety each joined by linker molecules. FIG. 1B is a schematic representation of one example of the schematic molecule embodiment illustrated in FIG. 1A. FIG. 1B shows how a polymer, poly(1-lysine hydrobromide) (40,000-60,000 kD), is conjugated to both a targeting moiety, goat anti-mouse IgG antibody, and an active moiety, lactoperoxidase, to form a DCTA molecule. The illustrated polymer is bound to a linker, sulfosuccinimidyl-4-(N-maleimidomethyl)cyclohexane-1-carboxylate, (S-SMCC), and the lactoperoxidase and antibody molecules are attached to the polymer using a modifier, N-succinimidyl S-acetylthioacetate (SATA). The three molecules are then combined to form a covalent bond between the maleimide group of the S-SMCC and the sulfhydryl group of SATA.

FIG. 2 is a series of gels and immunoblots confirming that the polymer poly(1-lysine hydrobromide) was successfully linked to goat anti-mouse IgG antibodies and lactoperoxidase molecules. FIG. 2A shows a Coomassie blue stained SDS-PAGE gel of polymer pellet and polymer supernatant samples. The high molecular weight bands indicate the linkage of lactoperoxidase (77.5 kDa) and/or goat anti-mouse IgG (150 kDa) to the polymer poly(1-lysine hydrobromide). FIGS. 2B and 2C show immunoblots following transfer of the gel samples in FIG. 2A onto a nitrocellulose membrane. The membrane was immunoblotted with either horseradish peroxidase (HRP)-conjugated rabbit anti-goat immunoglobulin (IgG) (FIG. 2B) or rabbit anti-lactoperoxidase IgG followed by HRP-conjugated mouse anti-rabbit IgG (FIG. 2C). Both blots indicate that the respective antibodies and lactoperoxidase enzyme were successfully linked to the polymer, and indicate that some free molecules not linked to the polymer were also present.

FIG. 3 is a series of photographs of immunohistochemical staining showing that the DCTA molecule recognizes protein targets in human tissue by immunohistochemical means. This confirms that the goat anti-mouse IgG antibody targeting moiety of the DCTA molecule is functional. FIG. 3A is a photograph showing the result of immunohistochemical staining using mouse anti-tropomyosin IgG, followed by goat anti mouse IgG. FIG. 3B is a photograph showing the result of immunohistochemical staining using rabbit anti-lactoperoxidase IgG, followed by goat anti-rabbit IgG. FIG. 3C is a photograph showing the result of immunohistochemical staining using mouse anti-tropomyosin IgG, followed by goat anti-rabbit IgG. FIG. 3D is a photograph showing the result of immunohistochemical staining using polymer supernatant, followed by goat anti-rabbit IgG. FIG. 3E is a photograph showing the result of immunohistochemical staining using mouse anti-tropomyosin IgG, followed by polymer supernatant, followed by goat anti-rabbit IgG. FIG. 3F is a photograph showing the result of immunohistochemical staining using polymer supernatant, followed by rabbit anti-lactoperoxidase, followed by goat anti-rabbit IgG. FIG. 3G is a photograph showing the result of immunohistochemical staining using tropomyosin IgG, followed by polymer supernatant, followed by rabbit anti-lactoperoxidase, followed by goat anti-rabbit IgG.

FIG. 4 is an autoradiograph showing that the DCTA molecule can label bovine serum albumin (BSA) protein with ¹²⁵I, confirming that the conjugated lactoperoxidase enzyme is functional in a solution containing exogenously added protein.

FIG. 5 shows that the DCTA molecule can label proteins extracted from prostate section with ¹²⁵I confirming that the conjugated lactoperoxidase enzyme functionally labels multiple proteins in a mixture from a tissue section.

FIG. 6 shows that the DCTA molecule can label proteins embedded in a prostate tissue section with ¹²⁵I, confirming that the conjugated lactoperoxidase enzyme is functional when added to a fixed tissue section.

FIG. 7 is a plot of the absorbance at 280 nm of protein (i.e., the maleimide-activated polymer) in twenty-seven elution fractions eluted from a 2-ml desalting column using 0.1 M sodium phosphate, 0.15 M NaCl, pH 7.2 as the elution buffer.

FIG. 8 is a series of photographs of immunohistochemical staining showing that the DCTA molecule stains tissue with cell specificity. FIG. 8A is a photograph showing the result of immunohistochemical staining using monoclonal mouse anti-E-cadherin IgG, followed by goat anti mouse IgG. FIG. 8B is a photograph showing the result of immunohistochemical staining using monoclonal anti-E-cadherin IgG, followed by polymer supernatant, followed by rabbit anti-lactoperoxidase, followed by goat anti-rabbit IgG. FIG. 8C is a photograph showing the result of immunohistochemical staining using polymer supernatant, followed by rabbit anti-lactoperoxidase antibodies, followed by goat anti-rabbit IgG antibodies. FIG. 8E is a photograph showing the result of immunohistochemical staining using monoclonal mouse anti-CD34 IgG followed by polymer supernatant, followed by rabbit anti-lactoperoxidase, followed by goat anti-rabbit IgG. FIG. 8F is a photograph showing the result of immunohistochemical staining using mouse anti-CD34 IgG, followed by goat anti-rabbit IgG antibodies.

### DETAILED DESCRIPTION

I. Abbreviations

DCTA: direct cell target analysis
DNA: deoxyribonucleic acid
ELISA: enzyme-linked immunosorbant assay
HRP: horseradish peroxidase
IgG: immunoglobulin G
LCM: laser capture microdissection
PBS: phosphate buffered saline
PCR polymerase chain reaction
RNA: ribonucleic acid
SATA: N-succinimidyl S-acetylthioacetate
SDS-PAGE: sodium dodecyl sulfate—polyacrylamide gel electrophoresis
S-SMCC: sulfosuccinimidyl-4-(N-maleimidomethyl)cyclohexane-1-carboxylate
   II. Terms

Unless otherwise noted, technical terms are used according to conventional usage. Definitions of common terms in molecular biology may be found in Benjamin Lewin, Genes V, published by Oxford University Press, 1994 (ISBN 0-19-854287-9); Kendrew et al. (eds.), The Encyclopedia of Molecular Biology, published by Blackwell Science Ltd., 1994 (ISBN 0-632-02182-9); and Robert A. Meyers (ed.), Molecular Biology and Biotechnology: a Comprehensive Desk Reference, published by VCH Publishers, Inc., 1995 (ISBN 1-56081-569-8).

In order to facilitate review of the various embodiments of the invention, the following explanations of specific terms are provided:

Amplification: Techniques that increases the number of copies of a molecule in a sample or specimen. An example of amplification of a nucleic acid molecule is the polymerase chain reaction, in which a biological sample collected from a subject is contacted with a pair of oligonucleotide primers, under conditions that allow for the hybridization of the primers to nucleic acid template in the sample. The primers are extended under suitable conditions, dissociated from the template, and then re-annealed, extended, and dissociated to amplify the number of copies of the nucleic acid. The product of in vitro amplification may be characterized by electrophoresis, restriction endonuclease cleavage patterns, oligonucleotide hybridization or ligation, and/or nucleic acid sequencing, using standard techniques. Other examples of in vitro nucleic acid amplification techniques include strand displacement amplification (see U.S. Pat. No. 5,744,311); transcription-free isothermal amplification (see U.S. Pat. No. 6,033,881); repair chain reaction amplification (see WO 90/01069); ligase chain reaction amplification (see EP-A-320 308); gap filling ligase chain reaction amplification (see U.S. Pat. No. 5,427,930); coupled ligase detection and PCR (see U.S. Pat. No. 6,027,889); and NASBA™ RNA transcription-free amplification (see U.S. Pat. No. 6,025,134).

Antibody: Immunoglobulin molecules and immunologically active portions of immunoglobulin molecules, i.e., molecules that contain an antigen binding site that specifically binds (immunoreacts with) an antigen.

A naturally occurring antibody (e.g., IgG, IgM, IgD) includes four polypeptide chains, two heavy (H) chains and two light (L) chains interconnected by disulfide bonds. However, it has been shown that the antigen-binding function of an antibody can be performed by fragments of a naturally occurring antibody. Antibody fragments that perform the antigen-binding function of an antibody are within the scope of the disclosure.

Immunoglobulins and certain variants thereof are known and many have been prepared in recombinant cell culture (e.g., see U.S. Pat. No. 4,745,055; U.S. Pat. No. 4,444,487; WO 88/03565; EP 256,654; EP 120,694; EP 125,023; Faoulkner et al., Nature 298:286, 1982; Morrison, J. Immunol. 123:793, 1979; Morrison et al., Ann Rev. Immunol 2:239, 1984).

Antibody-enzyme fusion: A chimeric fusion molecule that includes an antibody, or the variable binding domain of an antibody fused to an enzyme moiety.

Binding affinity: Binding affinity is a term that refers to the strength of binding of one molecule to another at a site on the molecule. If a particular molecule will bind to another particular molecule, these two molecules are said to exhibit binding affinity for each other. Binding affinity is related to the association constant and dissociation constant for a pair of molecules, but it is not critical to the disclosure that these constants be measured or determined. Rather, affinities as used herein refer to the specificity of the targeting moiety of the DCTA molecule for the cells or cell components of interest, i.e., whether the targeting moiety can specifically label these cells or cell components such that DCT analysis achieves sufficient separation or distinguishing of these molecules from their environment. The concepts of binding affinity, association constant, and dissociation constant are well known.

In certain embodiments, the binding affinity of the targeting moiety of the DCTA molecule is particularly high, as in the affinity of biotin for (strept)-avidin. In other embodiments, the affinity is simply sufficient to distinguish the cells or components of interest from their environment.

Binding domain: The molecular structure associated with that portion of a molecule that binds to another molecule. For example, the binding domain may comprise a polypeptide, natural or synthetic, or nucleic acid encoding such a polypeptide, the amino acid sequence of which represents a specific (binding domain) region of a protein, which either alone or in combination with other domains, exhibits binding characteristics that are the same or similar to those of a desired ligand/receptor binding pair, or an antibody/antigen pair (e.g., such as rabbit anti-lactoperoxidase, followed by goat anti-rabbit IgG). A nucleic acid molecule may also comprise a binding domain that exhibits binding characteristics (e.g., a nucleic acid probe that binds to complementary sequences in a sample). Neither the specific sequences nor the specific boundaries of such domains are critical, so long as binding activity is exhibited. Likewise, used in this context, binding characteristics include a range of affinities, avidities and specificities, and combinations thereof, so long as binding activity is exhibited.

Binding partner: Any molecule or compound capable of recognizing and binding to a specific structural aspect of another molecule or compound. Examples of such binding partners and corresponding molecule or compound include antigen/antibody, hapten/antibody, nucleic acid probe/complementary nucleic acid sequence, lectin/carbohydrate, apoprotein/cofactor and biotin/(strept)avidin.

Direct Cell Target (DCT) analysis (DCTA): The use of a Direct Cell Target Analysis molecule to distinguish or separate cells or cell components or molecules in a sample based upon a distinguishing characteristic or pattern of expression or distribution (e.g., of a protein, nucleic acid, or other molecule that is the target of the targeting moiety) within the sample. DCT analysis utilizes the pattern of expression or other distribution of a component as a means to distinguish or separate cells or components of cell from their environment (e.g., from within a tissue sample). Thus, the pattern of expression or distribution of molecules within cells or cell components, as recognized by the targeting moiety, enables a user to distinguish or separate the cells or cell components.

DCT analysis also can be accomplished by making a targeted component or cell uniquely visible or detectable within a sample, without or prior to removing the target from its surroundings. By way of example, DCT analysis can be accomplished by labeling a biomolecule in a way that causes the labeled target of interest to be distinguishable from its surroundings (e.g., by labeling the target with ¹²⁵I, such that only the labeled target is visualized when the radioactive signal is detected, for instance by autoradiography), or uniquely isolatable from its surroundings (e.g., by addition of an epitope or other purification or separation assistive component, such as a component of the strept/avidin:biotin system or another ligand/binding system).

Direct Cell Target Analysis (DCTA) molecule: A molecule having at least two functional moieties: a targeting moiety (also called a localizing moiety, because it localizes the DCTA molecule to a target cell or site within a sample), which targets the DCTA to specific component(s) (e.g., cells or structures within or upon those cells); and an active moiety, which facilitates the analysis of targeted components. In specific embodiments, these two moieties are attached to each other directly. In others, they are attached through a linker (for instance, a simple or a complex linker). Non-limiting examples of target and active moieties are disclosed herein.

DNA: DNA is a long chain polymer that comprises the genetic material of most living organisms (some viruses have genes comprising ribonucleic acid (RNA)). The repeating units in DNA polymers are four different nucleotides, each of which comprises one of the four bases, adenine, guanine, cytosine and thymine bound to a deoxyribose sugar to which a phosphate group is attached. Triplets of nucleotides (referred to as codons) code for each amino acid in a polypeptide. The term codon is also used for the corresponding (and complementary) sequences of three nucleotides in the mRNA into which the DNA sequence is transcribed.

Fluorophore: A chemical compound, which when excited by exposure to a particular wavelength of light, emits light (i.e., fluoresces), for example at a different wavelength than that to which it was exposed. Fluorophores can be described in terms of their emission profile, or “color.” Green fluorophores, for example Cy3, FITC, and Oregon Green, are characterized by their emission at wavelengths generally in the range of 515-540 nm (λ). Red fluorophores, for example Texas Red, Cy5 and tetramethylrhodamine, are characterized by their emission at wavelengths generally in the range of 590-690 nm (λ).

Encompassed by the term “fluorophore” as it is used herein are luminescent molecules, which are chemical compounds which do not require exposure to a particular wavelength of light to fluoresce; luminescent compounds naturally fluoresce. Therefore, the use of luminescent signals eliminates the need for an external source of electromagnetic radiation, such as a laser. An example of a luminescent molecule includes, but is not limited to, aequorin (Tsien, Ann. Rev. Biochem. 67:509, 1998).

Examples of fluorophores are provided in U.S. Pat. No. 5,866,366. These include: 4-acetamido-4′-isothiocyanatostilbene-2,2′disulfonic acid, acridine and derivatives such as acridine and acridine isothiocyanate, 5-(2′-aminoethyl)aminonaphthalene-1-sulfonic acid (EDANS), 4-amino-N-[3-vinylsulfonyl)phenyl]naphthalimide-3,5 disulfonate (Lucifer Yellow VS), N-(4-anilino-1-naphthyl)maleimide, anthranilamide, Brilliant Yellow, coumarin and derivatives such as coumarin, 7-amino-4-methylcoumarin (AMC, Coumarin 120), 7-amino-4-trifluoromethylcouluarin (Coumaran 151); cyanosine; 4′,6-diaminidino-2-phenylindole (DAPI); 5′,5″-dibromopyrogallol-sulfonephthalein (Bromopyrogallol Red); 7-diethylamino-3-(4′-isothiocyanatophenyl)-4-methylcoumarin; diethylenetriamine pentaacetate; 4,4′-diisothiocyanatodihydro-stilbene-2,2′-disulfonic acid; 4,4′-diisothiocyanatostilbene-2,2′-disulfonic acid; 5-[dimethylamino]naphthalene-1-sulfonyl chloride (DNS, dansyl chloride); 4-(4′-dimethylaminophenylazo)benzoic acid (DABCYL); 4-dimethylaminophenylazophenyl-4′-isothiocyanate (DABITC); eosin and derivatives such as eosin and eosin isothiocyanate; erythrosin and derivatives such as erythrosin B and erythrosin isothiocyanate; ethidium; fluorescein and derivatives such as 5-carboxyfluorescein (FAM), 5-(4,6-dichlorotriazin-2-yl)aminofluorescein (DTAF), 2′7′-dimethoxy-4′5′-dichloro-6-carboxyfluorescein (JOE), fluorescein, fluorescein isothiocyanate (FITC), and QFITC (XRITC); fluorescamine; IR144; IR1446; Malachite Green isothiocyanate; 4-methylumbelliferone; ortho cresolphthalein; nitrotyrosine; pararosaniline; Phenol Red; B-phycoerythrin; o-phthaldialdehyde; pyrene and derivatives such as pyrene, pyrene butyrate and succinimidyl 1-pyrene butyrate; Reactive Red 4 (Cibacron™ Brilliant Red 3B-A); rhodamine and derivatives such as 6-carboxy-X-rhodamine (ROX), 6-carboxyrhodamine (R6G), lissamine rhodamine B sulfonyl chloride, rhodamine (Rhod), rhodamine B, rhodamine 123, rhodamine X isothiocyanate, sulforhodamine B, sulforhodamine 101 and sulfonyl chloride derivative of sulforhodamine 101 (Texas Red); N,N,N′,N′-tetramethyl-6-carboxyrhodamine (TAMRA); tetramethyl rhodamine; tetramethyl rhodamine isothiocyanate (TRITC); riboflavin; rosolic acid and terbium chelate derivatives.

Other fluorophores include thiol-reactive europium chelates that emit at approximately 617 nin (Heyduk and Heyduk, Analyt. Biochem. 248: 216-227, 1997; J. Biol. Chem. 274: 3315-3322, 1999).

Other fluorophores include cyanine, merocyanine, styryl, and oxonyl compounds, such as those disclosed in U.S. Pat. Nos. 5,268,486; 5,486,616; 5,627,027; 5,569,587; and 5,569,766, and in published patent application PCT/US98/00475, each of which is incorporated herein by reference. Specific examples of fluorophores disclosed in one or more of these patent documents include Cy3 and Cy5, for instance.

Other fluorophores include GFP, Lissamine™, diethylaminocoumarin, fluorescein chlorotriazinyl, naphthofluorescein, 4,7-dichlororhodamine and xanthene (as described in U.S. Pat. No. 5,800,996 to Lee et al., herein incorporated by reference) and derivatives thereof. Other fluorophores are known to those skilled in the art, for example those available from Molecular Probes (Eugene, Oreg.).

Particularly useful fluorophores have the ability to be attached to (coupled with) a nucleotide, such as a modified nucleotide, are substantially stable against photobleaching, and have high quantum efficiency.

Functional fragments and variants of a polypeptide: Those fragments and variants that maintain one or more function(s) of the parent polypeptide, such as the binding activity of antibodies or antigen-binding fragments of antibodies to target cells. It includes any polypeptide eight or more, or 10 or more, or 15 or more, or 20 or more, or 25 or more residues in length that retains one or more function(s) of the parent polypeptide.

It is recognized that the gene or cDNA encoding a polypeptide can be considerably mutated without materially altering one or more of the polypeptide's functions. The genetic code is well known to be degenerate, and thus degenerate variants use different codons to encode the same amino acids. Contemplated herein are degenerate variants with at least 1, at least 2, at least 3, 5, 10, 15, or 20 or more degenerate changes compared to the parent. A conservative variant is a polypeptide in which an amino acid substitution is introduced, but the mutation can be conservative and have no material impact on at least one essential function of a protein (see Stryer, Biochemistry 3rd Ed., 1988). Contemplated herein are conservative variants containing at least 1, at least 2, at least 3, 5, 10, 15, or 20 or more changes compared to the parent protein.

In addition, part of a polypeptide chain can be deleted without impairing or eliminating all of its functions. Insertions or additions can also be made in the polypeptide chain, for example, adding epitope or other molecular tags, without impairing or eliminating one or more of its functions (Ausubel et al., J Immunol. 159(4): 1669-1675, 1997). Thus, a functional protein fragment could be modified using conservative substitutions of the sequences, yet retain its general function. In addition, a fragment can be derivatized to improve the biochemical stability of the fragment One specific, non-limiting example of a derivatized fragment is conjugation of the fragment to a conjugate including a polymeric backbone, such as polyethylene glycol (“PEG”), cellulose, dextran, agarose, or an amino acid copolymer (see U.S. Pat. No. 6,106,835). Such modifications are within the scope of the disclosure.

Other modifications that can be made without materially impairing one or more functions of a polypeptide include, for example, in vivo or in vitro chemical and biochemical modifications or incorporation of unusual or non-natural amino acids. Such modifications include, for example, acetylation, carboxylation, phosphorylation, glycosylation, ubiquitination, labeling, e.g., with radionuclides, and various enzymatic modifications, as will be readily appreciated by those well skilled in the art. These modifications that do not alter the function of fragments of a protein are within the scope of the disclosure. A variety of methods for labeling polypeptides and of substituents or labels useful for such purposes are well known in the art, and include radioactive isotopes such as ³²P, fluorophores, chemiluminescent agents, and enzymes.

Fusion molecule: A molecule that contains at least two component portions that do not naturally occur together in the same molecule. The term contemplates molecules that contain two different polypeptide portions (a fusion protein), two different nucleic acid portions (a fusion nucleic acid), at least one portion each of a polypeptide and a nucleic acid (a protein-nucleic acid fusion), and so forth. Other components that are contemplated as portions within a fusion molecule include, but are not limited to, organic and inorganic small molecules, cofactors, carbohydrates, fatty acids and fatty acid containing molecules, and so forth. The portions of a fusion molecule may be fused or joined to each other directly, or they may optionally be joined to each other through a linker. Non-limiting examples of linkers are described herein.

Fusion proteins have at least two domains or moieties fused (e.g., engineered through chemical, biochemical, or genetic engineering techniques) together, each portion of the protein comprising a region capable of independent structural or functional activity (i.e., forming a specific complex with a target molecule, or carrying out a biochemical reaction, for instance).

In some embodiments, the two domains of a fusion protein are either genetically fused together (e.g., nucleic acid molecules that encode each protein domain are functionally linked together) or chemically fused together (i.e., covalently bonded). By way of example, a fusion nucleotide may be produced such that it encodes both a targeting and an active moiety within a single fusion nucleotide molecule, with or without a linker oligonucleotide interposed there between. The translated product of such a fusion-encoding nucleic acid molecule (which is itself a fusion nucleic acid molecule) is the fusion protein. As used herein, examples of “fusion proteins” are proteins constructed to facilitate analysis of a tissue sample. Particular examples are DCTA molecules.

The two moieties of such a fusion protein are assembled in any order, or can each be linked independently to a separate linker molecule (e.g., a chemical crosslinker, a polymer complex, etc.). The moiety of the fusion molecule that targets the DCTA molecule to a target can be referred to as the targeting domain or targeting moiety, while the second moiety (which has a different activity) is the active domain or active moiety, and acts within or upon the target component to generate a detectable signal. The domains/moieties need not be organized in a specific order; the amino-proximal domain of the fusion protein may be either the localizing domain or the active domain; likewise for the carboxy-proximal domain.

Fusion molecules can be further characterized according to the target they bind to and/or act upon. For instance, a fusion molecule that binds to specific sites on kidney glomeruli may be referred to as a glomeruli-targeted fusion molecule (or specifically, a glomeruli-targeted DCTA).

Hybridization: Oligonucleotides hybridize by hydrogen bonding, which includes Watson-Crick, Hoogsteen or reversed Hoogsteen hydrogen bonding between complementary nucleotide units. For example, adenine and thymine are complementary nucleobases that pair through formation of hydrogen bonds. “Complementary” refers to sequence complementarity between two nucleotide units. For example, if a nucleotide unit at a certain position of an oligonucleotide is capable of hydrogen bonding with a nucleotide unit at the same position of a DNA or RNA molecule, then the oligonucleotides are complementary to each other at that position. The oligonucleotide and the DNA or RNA are complementary to each other when a sufficient number of corresponding positions in each molecule are occupied by nucleotide units which can hydrogen bond with each other. Hybridization can occur between sequences either in vitro or in vivo, and can occur between an in vivo sequence and an externally added component, such as the DCTA molecule that comprises a nucleic acid targeting moiety.

Hybridization conditions resulting in particular degrees of stringency will vary depending upon the nature of the hybridization method of choice and the composition and length of the hybridizing DNA used. Generally, the temperature of hybridization and the ionic strength (especially the Na⁺ concentration) of the hybridization buffer will determine the stringency of hybridization. Calculations regarding hybridization conditions required for attaining particular degrees of stringency are discussed by Sambrook, Fritsch, and Maniatis, Molecular Cloning: a Laboratory Manual, 2^{nd} Edition, Cold Spring Harbor Laboratory Press, USA, (1989);

Isolated: An “isolated” biological component (such as a nucleic acid molecule, protein or organelle) has been substantially separated or purified away from other biological components in the environment in which the component naturally occurs, i.e., other chromosomal and extra-chromosomal DNA and RNA, proteins and organelles. By way of example, a component can be isolated from a cell, a biochemical reaction mixture, and so forth.

Lactoperoxidase-mediated iodination: The use of lactoperoxidase to iodinate (add an iodine atom to) proteins. For example, lactoperoxidase can be used to iodinate residues in a protein and ¹²⁵I label can be incorporated into the protein during this labeling process (see, for instance, Thorell and Johansson, Biochim. Biophys. Acta, 251(3): 363-369, 1971; Courtoi & Hughes, Gerontology 22: 371-739, 1976; and Sun and Dunford, Biochemistry 32(5): 1324-1331, 1993). Known lactoperoxidase enzymes include but are not limited to those encoded by GenBank Accession No. BC016212, XM_{—}042207, NM_{—}173933, NM_{—}080420, AK018070, AF498045, AJ131675, AF027971, BF454677, AF027970, M58151, and M58150.

Linker: A linker is a “chemical arm” between two moieties or domains in a molecule. Linkers may be used to join otherwise separate molecule moieties through a chemical reaction. The term “linker” also refers to the part of a DCTA molecule between two moieties or subsections. In some embodiments, the linker in a DCTA molecule is added by recombinant DNA techniques; in other embodiments, it is added through chemical means, such as crosslinking reactions or other in vitro chemical synthesis.

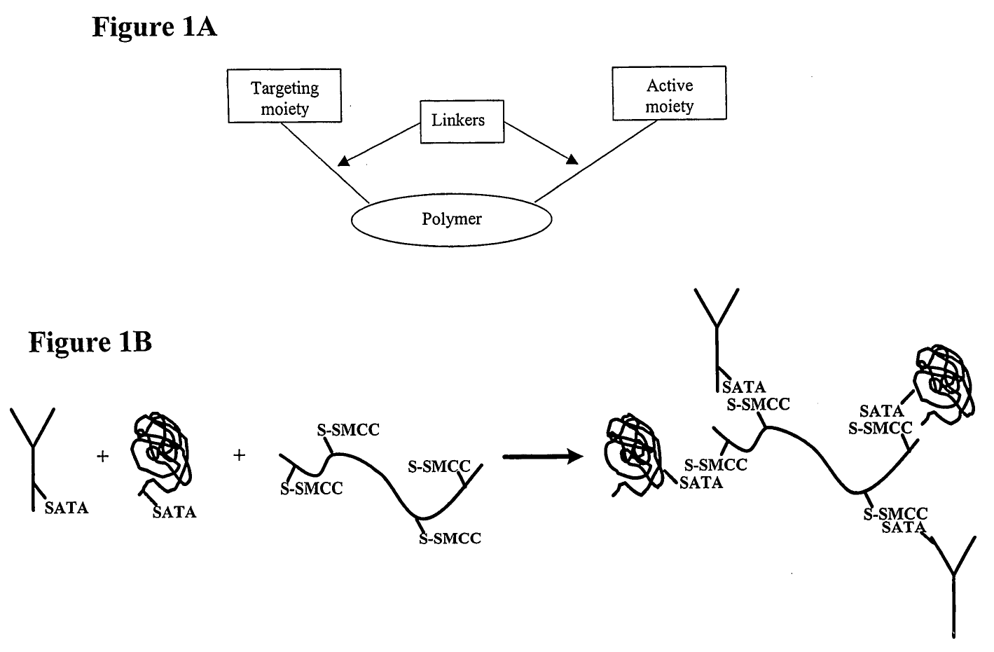

Many sorts of different chemical structures may constitute a linker (e.g., a peptide-to-peptide bond, a covalent bond between two protein domains, such as an amide, ester, or alkylamino linkages, or a single translated protein having two moieties “linked” by a series of residues). One non-limiting example of a linker is a synthetic sequence of amino acids. An example of a specifically contemplated complex linker (as illustrated in FIG. 1) comprises a polymer molecule, which serves as the central structural molecule; and one or more simple linker components, which connect the polymer to the targeting and active moieties. Other examples of linkers include streptavidin linkage, a straight or branched chain aliphatic group, particularly an alkyl group, such as C₁-C₂₀, optionally containing within the chain double bonds, triple bonds, aryl groups or heteroatoms such as N, O or S. Substituents on a diradical moiety can include C₁-C₆ alkyl, aryl, ester, ether, amine, amide, or chloro groups.

Some linkers are complex, in that they are made of more than one component. An example of a complex linker, and the construction of a DCTA molecule comprising a complex linker, is shown in FIGS. 1A and 1B. In the illustrated embodiment of a DCTA molecule, the complex linker includes a polymer poly(1-lysine hydrobromide), of approximately 40,000-60,000 kDa, joined to both a targeting moiety (goat anti-mouse IgG antibody) and an active moiety (lactoperoxidase) by covalent bonds created through the use of simple linker molecules (illustrated here with sulfosuccinimidyl-4-N-maleimidomethyl)cyclohexane-1-carboxylate in FIG. 1B). In FIG. 1B, further chemicals are used to catalyze formation of chemical linkages between the elements of the complex linker (illustrated with the catalyzing molecule modifier S-acetylthioglycolic acid N-hydroxysuccinimide ester). An alternative complex linker is the combination of strept/avidin with biotin; these molecules can be attached to two protein domains, and the interaction between the strept/avidin:biotin pair serves to link the domains of the fusions.

It is specifically contemplated that fusion molecules as provided herein can contain multiple copies of active moieties, and/or multiple copies of targeting moieties. A representative example of such complex DCTA molecules is shown in FIG. 1B.

Additional types of bond combinations that may serve to link molecules are amino with carboxyl to form amide linkages, carboxy with hydroxy to form ester linkages or amino with alkyl halides to form alkylamino linkages, thiols with thiols to form disulfides, thiols with maleimides, and alkylhalides to form thioethers, for instance. Hydroxyl, carboxyl, amino and other functionalities, where not present may be introduced by known methods. Examples of specific linkers can be found, for instance, in Hennecke et al. (Protein Eng. 11: 405-410, 1998); and U.S. Pat. Nos. 5,767,260 and 5,856,456.

Linkers may vary in length in different embodiments, depending for instance on the molecular moieties being joined, on their method of synthesis, and on the intended function(s) of the DCTA molecule.

Linkers may be repetitive or non-repetitive. One classical repetitive peptide linker used in the production of single chain Fvs (SCFvs) is the (Gly₄Ser)₃ (or (GGGGS)₃ or (G₄S)₃) linker. More recently, non-repetitive linkers have been produced, and methods for the random generation of such linkers are known (Hennecke et al, Protein Eng. 11: 405-410, 1998). In addition, linkers may be chosen to have more or less secondary character (e.g. helical character, U.S. Pat. No. 5,637,481) depending on the conformation desired in the final fusion molecule. The more secondary character a linker possesses, the more constrained the structure of the final fusion molecule will be. Therefore, substantially flexible linkers that are substantially lacking in secondary structure allow flexion of the fusion molecule at the linker.

Microdissection: A process of isolating an element from a sample, often a tissue sample, at a microscopic level.

Moiety: A part or portion of a molecule having a characteristic chemical, biochemical, structural and/or pharmacological property or function. As used herein, the term moiety refers to a subpart of a molecule that retains an independent biochemical or structural activity from the remainder of the molecule, for instance the ability to generate heat or fluoresce, or to bind or associate with a target or to carry out an enzymatic reaction. A single molecule may have multiple moieties, for instance an antigen-binding (e.g., antibody or antibody-derived) moiety and a transcriptase moiety, each having an independent function. Additionally, a moiety of a molecule may be activated at different times and by different catalysts, for instance by addition of a reagent, a change of temperature, or reaction with light.

Oligonucleotide and oligonucleotide analogs: A plurality of nucleotides joined by phosphodiester bonds between about 6 and about 300 nucleotides in length. An oligonucleotide analog is a molecule that functions similarly to an oligonucleotide but has one or more non-naturally occurring portions. For example, oligonucleotide analogs can contain non-naturally occurring portions, such as altered sugar moieties or inter-sugar linkages, such as a phosphorothioate oligodeoxynucleotide. Functional analogs of naturally occurring polynucleotides can bind to RNA or DNA, and include peptide nucleic acid (PNA) molecules.

Particular oligonucleotides and oligonucleotide analogs include linear sequences up to about 300 nucleotides in length, for example a sequence (such as DNA or RNA) that is at least 6 bases, for example at least 8, 10, 15, 20, 25, 30, 35, 40, 45, 50, 100, 150, 200 or even 300 bases long, or from about 6 to about 50 bases, for example about 10-25 bases, such as 12, 15 or 20 bases.

Oligonucleotide-enzyme fusion: A fusion molecule comprising a hybridizable nucleic acid fused to an enzyme or functional fragment thereof. This is a specific category of protein-nucleic acid fusion molecules.

Operably linked: A first molecule (e.g., nucleic acid sequence, protein, linker, etc.) is operably linked with a second molecule when the first nucleic acid sequence is placed in a functional relationship with the second nucleic acid sequence. For instance, a promoter is operably linked to a coding sequence if the promoter affects the transcription or expression of the coding sequence. Generally, operably linked DNA sequences are contiguous and, where necessary to join two protein-coding regions, in the same reading frame.

Polymer: Polymers are substances (e.g., protein, nucleic acid sequences) consisting of large molecules that are made of many small, repeating units called monomers. The number of repeating units in one large molecule is called the degree of polymerization. A polymer may be joined with other molecules to form a complex linker, as in the DCTA molecule of the disclosure. Polymers that are suitable for use in the disclosure may have different features (e.g., polyamine, polycarboxylate, polystyrene, etc.), and may range in size.

Primers: Primers are short nucleic acid molecules, preferably 10 nucleotides or more in length. In some embodiments, longer primers can be about 15, 17, 20, or 23 nucleotides or more in length. Nucleic acid primers can be readily prepared based on a nucleic acid sequence. Primers can be annealed to a complementary target nucleic acid (DNA or RNA) by nucleic acid hybridization to form a hybrid between the primer and the target nucleic acid, and then the primer extended along the target nucleic acid by a DNA polymerase enzyme. Primer pairs can be used for amplification of a nucleic acid sequence, e.g., by the polymerase chain reaction (PCR) or other nucleic-acid amplification methods.

Methods for preparing and using primers are described, for example, in Sambrook, Fritsch, and Maniatis, Molecular Cloning: a Laboratory Manual, 2^{nd} Edition, Cold Spring Harbor Laboratory Press, USA, (1989); Ausubel et al. (In Current Protocols in Molecular Biology, Greene Publ. Assoc. and Wiley-Intersciences, 1998), and Innis et al. (PCR Protocols, A Guide to Methods and Applications, Academic Press, Inc., San Diego, Calif., 1990). PCR primer pairs can be derived from a known sequence, for example, by using computer programs intended for that purpose such as Primer (Version 0.5, © 1991, Whitehead Institute for Biomedical Research, Cambridge, Mass.). One of ordinary skill in the art will appreciate that the specificity of a particular primer increases with its length. Thus, for example, a primer comprising 30 consecutive nucleotides will anneal to a target sequence with a higher specificity than a corresponding primer of only 15 nucleotides. Thus, in order to obtain greater specificity, primers can be selected that comprise at least 17, 20, 23, 25, 30, 35, 40, 45, 50 or more consecutive nucleotides.

Probes: An isolated nucleic acid attached to a detectable label or other reporter molecule. Typical labels include radioactive isotopes, enzyme substrates, co-factors, ligands, chemiluminescent or fluorescent agents, haptens, and enzymes. Methods for labeling and guidance in the choice of labels appropriate for various purposes are known, e.g., Sambrook et al. (In Molecular Cloning: A Laboratory Manual, CSHL, New York, 1989) and Ausubel et al. (In Current Protocols in Molecular Biology, John Wiley & Sons, New York, 1998).

Protein: A biological molecule expressed by an encoding nucleic acid (e.g., a gene or cDNA) and comprised of amino acids.

Purified: The term “purified” does not require absolute purity; rather, it is intended as a relative term. Thus, for example, a purified protein preparation is one in which the protein referred to is more pure than the protein in its natural environment within a cell or within a production reaction chamber (as appropriate). A purified nucleic acid is one in which the nucleic acid is more pure than in its natural environment within a cell or within a production chamber (as appropriate). Likewise, a purified organelle preparation is one in which the specified organelle is more pure than in its natural environment within a cell, so that only relatively insubstantial amounts (e.g. less than 10% relative) of other organelles (or markers for other organelles) are present in the preparation.

Recombinant: A recombinant nucleic acid is one that has a sequence that is not naturally occurring or has a sequence that is made by an artificial combination of two otherwise separated segments of sequence. This artificial combination can be accomplished by chemical synthesis or, more commonly, by the artificial manipulation of isolated segments of nucleic acids, e.g., by genetic engineering techniques.

RNA: A polymer formed from covalently linked ribonucleotide monomers. The repeating units in RNA polymers are four different nucleotides, each of which comprises one of the four bases, adenine, guanine, cytosine and uracil bound to a ribose sugar to which a phosphate group is attached.

Separate(d)/Separation: To spatially dissociate components, such as biomolecules, cells, or cell clusters, from their surrounding environment and/or from each other. Separation may be employed, for instance, to analyze the characteristics of the separated components.

Some components (for example, proteins or peptides) can readily be separated based on one or more specific characteristics, such as molecular weight or mass, charge or isoelectric point, conformation, association in a complex, and so forth. Separation may be accomplished by any number of techniques, such as sucrose gradient centrifugation, aqueous or organic partitioning (e.g., 2-phase partitioning), non-denaturing gel electrophoresis, isoelectric focusing gel electrophoresis, capillary electrophoresis, isotachyphoresis, mass spectroscopy, chromatography (e.g., HPLC), polyacrylamide gel electrophoresis (PAGE, such as SDS-PAGE), and so forth.

Separation may also be accomplished by dissection of components of interest away from their environment. Dissection can be on various scales, for instance large-scale physical dissection wherein the operator visualizes the process, and small-scale or microdissection wherein the operator uses a visual aid (e.g., microscope) to accomplish the dissection. Separation by dissection can be accomplished by physical (e.g., manual) dissection or by use of the biochemical means (e.g., use of the characteristic(s) of the sample or exogenously added molecules to effect the dissection) as described herein. By way of example, a DCTA molecule containing an antibody targeting moiety and a biotin active moiety can be applied to a sample and allowed to localize in cells containing the target antigen. Following the localization step, the sample is solubilized and applied to a column containing bound strept/avidin, followed by washes with buffered solution. As only the molecules having biotin will remain bound to the column, those molecules are “dissected” away from the remainder of the sample.

Separation is a relative rather than an absolute term (in that separation need not be perfect or “complete” for components to be “separated”). Thus, when a sample is subjected to a separation technique and the resultant separated sample is divided into fractions (e.g., fractions from a sucrose gradients or purification column, bands from a gel, and so forth), components within the sample can still be referred to as “separated” even though they occur in more than one of the fractions. Similarly, a certain cell type can be separated from a tissue sample without requiring that it be entirely purified from other cell types (e.g., “dissected” as discussed above).

In some instances, separation of components from each other or from their environment is accomplished by employing a separation (or purification) assistive component. It is contemplated that components of a recognized specific binding partner system can be used as separation assistive components. Specific binding partners include molecules or compositions capable of recognizing and binding to a specific structural aspect of another molecule or composition. Examples of such binding partners and corresponding molecules or compositions include antigen/antibody, hapten/antibody, lectin/carbohydrate, apoprotein/cofactor and strept/avidin:biotin. Thus, separation assistive components include, but are not limited to: epitopes (which enable separation based on specific recognition by an antibody), components of the strept/avidin:biotin system (which enable separation based on recognition by another component in that system), a component in another ligand/binding system, and so forth. Many other examples of specific binding partner systems are well known to those of ordinary skill in the art.

Specific binding agent: An agent that binds substantially only to a defined target Thus a protein-specific binding agent binds substantially only the defined protein, or a peptide region within a protein. As used herein, the term “specific binding agent,” refers to a specific protein or peptide, including antibodies (and functional fragments thereof) and other agents (such as soluble receptors) that bind substantially only to target proteins. In some embodiments, these target proteins are within target cells of interest.

Antibodies may be produced using standard procedures described in a number of texts, including Harlow and Lane (Antibodies, A Laboratory Manual, CSHL, New York, 1988). The determination that a particular agent binds substantially only within the target cells may readily be made by using or adapting routine procedures. One suitable in vitro assay makes use of the Western blotting procedure (described in many standard texts, including Harlow and Lane, Antibodies, A Laboratory Manual, CSHL, New York, 1988). Western blotting may be used to determine that a given protein binding agent binds substantially only to the specified protein.

Shorter fragments of antibodies can also serve as specific binding agents. For instance, FAbs, Fvs, and single-chain Fvs (SCFvs) that bind to a protein or peptide within a target cells would be target cell-specific binding agents. These antibody fragments are defined as follows: (1) FAb, the fragment that contains a monovalent antigen-binding fragment of an antibody molecule produced by digestion of whole antibody with the enzyme papain to yield an intact light chain and a portion of one heavy chain; (2) FAb′, the fragment of an antibody molecule obtained by treating whole antibody with pepsin, followed by reduction, to yield an intact light chain and a portion of the heavy chain; two FAb′ fragments are obtained per antibody molecule; (3) (FAb′)₂, the fragment of the antibody obtained by treating whole antibody with the enzyme pepsin without subsequent reduction; (4) F(Ab′)₂, a dimer of two FAb′ fragments held together by two disulfide bonds; (5) Fv, a genetically engineered fragment containing the variable region of the light chain and the variable region of the heavy chain expressed as two chains; and (6) single chain antibody (“SCA”), a genetically engineered molecule containing the variable region of the light chain, the variable region of the heavy chain, linked by a suitable polypeptide linker as a genetically fused single chain molecule. Methods of making these fragments are routine.

Specific binding partner: A member of a pair (or system) of molecules that interact by means of specific, noncovalent interactions that depend on the three-dimensional structures of the molecules involved. Typical sets of specific binding partners include antigen (or epitope)/antibody, hapten/antibody, hormone/receptor, nucleic acid strand/complementary nucleic acid strand, substrate/enzyme, inhibitor/enzyme, carbohydrate/lectin, biotin/(strept)avidin, and virus/cellular receptor.

Subject: Living multi-cellular vertebrate organisms, a category that includes both human and non-human mammals.

Support: As used herein, the term “support,” refers to the ability of an environment (e.g., cell, tissue section, etc.) to provide all components necessary at a sufficient local level to conduct the activity of an active moiety. It is contemplated that one or more necessary components can be added to an environment to enable it to support a particular reaction. A reaction is “supported” within a cell or other reaction vessel when the components necessary to carry out the reaction are present. Thus, a cell supports a biochemical reaction, such as transcription, if the components necessary for transcription (i.e., template, nucleotides, enzymes, transcription factors, temperature, etc.) are present within the cell.

Unless otherwise explained, all technical and scientific terms used herein have the same meaning as commonly understood by one of ordinary skill in the art to which this invention belongs. The singular terms “a,” “an,” and “the” include plural referents unless context clearly indicates otherwise. Similarly, the word “or” is intended to include “and” unless the context clearly indicates otherwise. “Comprising” means “including.” It is further to be understood that all base sizes or amino acid sizes, and all molecular weight or molecular mass values, given for nucleic acids or polypeptides are approximate, and are provided for description. Although methods and materials similar or equivalent to those described herein can be used in the practice or testing of the present invention, suitable methods and materials are described below. All publications, patent applications, patents, and other references mentioned herein are incorporated by reference in their entirety. In case of conflict, the present specification, including explanations of terms, will control. In addition, the materials, methods, and examples are illustrative only and not intended to be limiting.

III. Description of Several Specific Embodiments

Provided herein are methods of Direct Cell Target (“DCT”) analysis, which are methods of analyzing a tissue sample(s) using a DCTA molecule. DCTA molecules comprise a targeting moiety capable of localizing the DCTA molecule to target cells or components of a cell within the sample, and an active moiety capable of generating a detectable signal or product.

DCT methods for sample analysis include: contacting a DCTA molecule with the tissue sample under conditions that allow at least a portion of the DCTA molecule to interact with at least a portion of the tissue sample, thereby allowing the DCTA molecule to localize to targeted cells or components within the sample; activating the active moiety of the DCTA molecule; and detecting the signal or product generated by the activated second moiety, thereby analyzing the sample. The methods provided are useful in analyzing a variety of samples, including but not limited to biopsy material, tissue sections, cell culture preparations, cytology preparations, cells grown in vitro, and cells grown in vivo.

Representative examples of a DCTA molecule comprise an antibody binding domain, a ligand, a DNA probe, or a primer as a targeting moiety. Additional examples use a DNA polymerase, lactoperoxidase, or reverse transcriptase as an active moiety. In embodiments in which the active moiety is lactoperoxidase, the detectable products generated include iodinated tryptophan and/or tyrosine residues. In a specific embodiment, ¹²⁵I is used to label the tryptophan and/or tyrosine residues, thereby creating a detectable signal in the form of radioiodinated peptides or proteins.

In another specific embodiment, the DCTA molecule comprises a targeting moiety and an active moiety covalently linked to a polymer. In a specific example of this embodiment, the DCTA molecule includes a poly(1-lysine hydrobromide) polymer as a linker, conjugated to lactoperoxidase and a targeting moiety. In specific representative examples of such molecules, the targeting moiety is an antibody or binding fragment thereof, such as for instance a goat anti-mouse IgG antibody. In examples where the binding moiety comprises a “standardized” binding domain, the DCTA molecule can be used to detect multiple different targets. A representative standardized targeting moiety is a “secondary” antibody or antibody binding domain, such as an anti-IgG antibody, where the antibody recognizes IgG produced in a different species than that used to produce the primary antibody.

In some DCT methods, one or more components necessary to generate a detectable signal are externally provided, such as components necessary for generation of detectable products in embodiments in which the active moiety is reverse transcriptase or DNA polymerase.

The disclosure further provides methods of screening for disease in a sample from a subject using DCT analysis, which methods involve screening for a protein or a nucleic acid encoding that protein, wherein the production of that protein by the target tissue is indicative of disease in the subject.

In a specific embodiment, DCT analysis is used to compare levels of expression of a nucleic acid, as compared to a control nucleic acid, wherein the elevated or decreased expression is indicative of disease in the subject In a specific embodiment, DCT analysis is used to screen for a nucleic acid(s) that is elevated in neoplasia.

Additional methods are provided in which DC analysis is used to screen for a disease in which the absence of the nucleic acid in the target cells is indicative of disease in the subject.

Further methods disclosed include the use of DCT analysis to screen for a disease in which the absence of the hormone is indicative of disease in the subject.

Additional methods are provided in which DCT analysis is used to screen for the presence of a mutation in a nucleic acid of the target cells from a subject, wherein the presence of such a mutation is indicative of disease.

Also provided are methods for automation of DCT analysis, in which all or a portion of the DCT analysis method is automated.

In still other embodiments, the detectable products are amplified, either in vivo or following removal from the tissue sample.

The disclosure further provides kits for use in DCT analysis methods. Specific examples of such kits provide a DCTA molecule (or one or more components for synthesis of the DCTA molecule) and/or one or more agents for use in the tissue analysis. In one specific embodiment, the kit contains a DCTA molecule capable of targeting cells of interest in the sample and producing a detectable biological signal. In this embodiment, the detected signal provides information regarding whether a biological condition is present.

In other embodiments, the kit includes a DCTA molecule capable of targeting cells of interest in the sample and producing a detectable biological signal, which provides information regarding whether the subject has a biological condition. In example kits, the detected components may be removed for subsequent analysis and may, optionally, be quantified. In other example kits, the signal may be detected without physical removal of any of the detected products.

IV. Methods of Direct Cell Target Analysis

Tissue analysis involves the selection of components of interest from a sample of tissue for focused study. However, under a microscope, tissues are heterogeneous, complicated structures with hundreds of different cell types. Thus, methods of distinguishing specific cells or cell components from specific microscopic regions of tissue sections or procuring components from their complex environment, are helpful to facilitate further molecular analysis methods in the study of cell components of interest.

Disclosed herein are methods of utilizing biochemical characteristic(s) of exogenously added fusion molecules to achieve non-mechanical, in situ target analysis of specific cells or cell components in a sample. Using these methods, the targeted biomolecules can be directly visualized in subsequent molecular analyses or procured and selectively analyzed (e.g., radioactive labeling).

In overview, Direct Cell Target Analysis (DCTA) molecules are provided that localize within or upon a cell or subset of cells of interest in a tissue sample (i.e., “target cells”) based upon the binding activity of a targeting portion (hereafter, “targeting moiety”) of the molecule. After localization, a second moiety of the DCTA molecule (hereafter, “active moiety”), acts upon or within the targeted cells to generate a detectable signal, thereby facilitating the targeted analysis. Thus, Direct Cell Target (“DCT”) analysis methods enable distinguishing or separation of target components (such as cells that express a specific protein, such as a surface antigen, for example a protein associated with disease) from their surrounding environment based upon their pattern of expression or distribution within the sample. This targeted analysis can be accomplished either with and without the component(s) of interest being physically separated from the heterogeneous tissue environment. Methods of targeting DCTA molecules to specific cell types within a tissue sample are provided, as are mechanisms of effecting the DCT analysis.

Using DCT analysis, functional genomics and proteomics can be brought down to the level of individual cells in a tissue. Analysis of protein and mRNA levels within specific cells and tissue structures will help determine whether and to what extent genes are operative in normal versus diseased cells. Isolation of specific cells will make it possible to detect somatic mutations in cellular DNA that result in malignancy. The disclosed methods can be used to follow changes in gene expression that accompany cell maturation, tumorigenesis, and cell apoptosis. Furthermore, the identification of specific protein products produced by diseased cells may provide information that can be used to develop new diagnostic methods to scan for the presence of such proteins. Each of these things can be advanced by DCT analysis methods, which enable selective identification and/or isolation of specific cells or cellular components such as DNA, RNA, and proteins, and mRNA from tissue samples. With a unified picture of the DNA structure, overall and specific RNA levels, and protein levels in particular cells, the molecular nature of many disease states can be better understood.

DCT analysis can be used to eliminate physical microdissection entirely from the process of molecular analysis of specific cell types in a heterogeneous population. Additionally, because DCT analysis eliminates the time-consuming step of physical removal of cells from a complex cell population, DCT analysis can be used to analyze a much larger number of cells than Laser Capture Microdissection or manual microdissection methods. For example, a user can perform DCT analysis on a large number (e.g., 30-40) histology slides, each containing several hundred thousand cells of interest, in one or two hours. Thus, DCT analysis allow for the analysis of many millions of cells in a few hours, when procurement of the same number of cells using traditional microdissection techniques would require days or weeks.

Furthermore, due to the specificity of the DCTA molecule for cells or cell components of interest, DCT analysis methods reduce the number of cells needed to obtain molecular profiling information in comparison to existing molecular profiling systems. In addition, DCTA molecules can be produced and used in quantified amounts and/or applied using automation, leading to more uniform and reproducible results than those achieved using manual or laser microdissection.

DCT analysis methods provide flexibility in the choice of targeting and active moieties, allowing the user to adapt the system to the sample being studied and the detection resources available to the user. Furthermore, DCT analysis optionally can be performed using automated machinery, eliminating the need for a human operator, and is applicable for use in many cell preparations, including tissue sections, cells in culture, cytology preps, or cells in vivo.

A. Construction of Direct Cell Target Analysis Molecules

DCTA molecules are constructed to combine two functional domains, or “moieties” in a single molecule. A “targeting moiety” is combined with an “active moiety” such that application of separate components is not necessary. The targeting moiety targets the DCTA molecule to the target cells or cellular components, and ensures that only those cells that are targeted are the ones acted upon by the active moiety. In some embodiments, a linker molecule is provided, to join the respective moieties.

DCTA molecules can be assembled in any order, and can be tailored to the types of cells and cell components a user desires to study as well as the detection methods available to the user. By way of example, the targeting moiety can be linked directly to the active moiety, or the molecule can be assembled by linking both moieties independently to a separate molecule, such as a polymer. In some embodiments, the DCTA molecule consists of one targeting molecule and one active molecule joined to a single polymer. In other embodiments, two or more targeting or active moieties are linked to a single polymer to increase the ability of the DCTA molecule to bind to and act upon its target In some embodiments, different numbers of targeting and active moieties are linked to a polymer molecule, and optionally, are linked using linker and/or modifier molecules.

The construction of chimeric molecules as fusion proteins from domains of known proteins is well known In general, nucleic acid molecules that encode the desired DCTA molecule are joined using standard techniques to create a single, operably linked fusion oligonucleotide, including recombinant DNA techniques. Molecular biological techniques may be found in Sambrook et al. (In Molecular Cloning: A Laboratory Manual, Cold Spring Harbor, N.Y., 1989).

By way of example, a DCTA molecule can be created using recombinant techniques by cloning nucleic acid sequences encoding a polymer, a targeting moiety, an active moiety, and linkers into an expression vector. Following induction of the expression vector, a protein is synthesized and purified using techniques known to those of skill in the art, such as those found in Sambrook et al. (In Molecular Cloning: A Laboratory Manual, Cold Spring Harbor, N.Y., 1989). In some embodiments, the targeting moiety is a single-chain antibody: although the H and L chains of an Fv fragment are encoded by separate genes, a synthetic linker can be made that enables them to be made as a single protein chain by recombinant methods; see Bird et al. Science 242: 423426, 19888; and Huston et al. PNAS 85: 5879-5883, 1988).

Alternatively, a DCTA molecule may be synthesized using traditional chemical synthesis techniques, as found in Foulon et al., Bioconjug Chem, 10(5): 867-76, 1999. An example of a method of synthesis of a DCTA molecule is shown in FIG. 1B. In this example, a targeting moiety (indicated as an antibody, e.g., goat anti-mouse IgG) and an active moiety (indicated as a two-dimensional protein enzyme, e.g., lactoperoxidase) are each attached to a modifier N-succinimidyl S-acetylthioacetate (SATA). The two types of molecules are combined in the presence of a polymer poly(1-lysine hydrobromide), which has linker molecules sulfosuccinimidyl-4-(N-maleimidomethyl)cyclohexane-1-carboxylate (S-SMCC) attached at multiple points along the polymer. Combination of these molecules under conditions set forth in Example 1, for instance, results in assembly into a complex linker molecule. FIG. 1B is a structural diagram of one potential embodiment, though it should not be construed as limiting. For example, in some embodiments, multiple active and/or targeting moieties can be joined to a single polymer, or can be directly joined to each other.

### Selection of Targeting Moiety

The selection of a population of cells for study influences the types of targeting moiety to be used Essentially any cell type is suitable for targeting with DCT analysis, where a molecule is known or can be identified that is suitable as a cell-specific target molecule to which the targeting moiety of the DCTA molecule can be directed.

Certain embodiments contemplate the use of antibodies (or fragments thereof) as targeting moieties. In some embodiments, the targeting moiety is an antibody (e.g., rabbit anti-goat IgG, for example) directed to bind a specific protein in a tissue preparation. In such embodiments, DCT analysis extends the ability to isolate pure populations of immunotypically defined cells from a sea of similarly appearing cells, and process such cells for further analysis.

In certain embodiments, the targeting moiety comprises a hapten, a lectin, a carbohydrate, a cofactor, a receptor ligand, or a protein with high specificity for a binding partner, such as the biotin/(strept)avidin binding pair, or protein A or G.

In other embodiments, a primer or DNA sequence serves as the targeting moiety, and binds to a complementary sequence within the target cells. By way of non-limiting example, the target is an expressed sequence such as a transcription factor, or a DNA regulatory sequence. In certain embodiments, the complementary sequence to which the targeting moiety binds/hybridizes (the “target”) is present in the cell or sample as a result of ongoing transcription. Thus, DCT analysis in these embodiments may be used to profile the response of target cells to internal or external conditions (e.g., treatments with pharmaceuticals, onset of disease, etc.). In some embodiments, the target primers are used as components in in vivo nucleic acid amplification (e.g., semi-quantitative RT-PCR) for subsequent analysis of the amplified products.

### Selection of Optional Linker Molecule(s)

Optionally, a linker is used to join the targeting moiety to the active moiety of the DCTA molecule. The choice of linker used may be influenced by the targeting or active moieties, the type of sample to be analyzed, and the targeting site selected. Linkers may be added through chemical or recombinant technology methods.

In general, the linker used in any DCTA molecule is of a length and secondary character to hold the active moiety within proximity of the target cell or cell structure after the targeting moiety has interacted with its target molecule. Linkers of varying type, length, and composition are within the scope of the disclosure.

Specific moieties can be lied using known chemical linking techniques, including chemical cross-linking. Cross-linkers are well known, and examples of molecules used for cross-linking can be found, for instance, in U.S. Pat. No. 6,027,890 (“Methods and compositions for enhancing sensitivity in the analysis of biological-based assays”) and Hermanson, Bioconjugate Techniques, (Academic Press, San Diego, Calif., 1996).

In specific embodiments, a simple chemical linker is attached to a polymer through biochemical or other means. Next, a “modifier” is used to attach one or more copies of the targeting moiety that is specific for certain cells or cell types and one or more copies of the active moiety, to the polymer. In one example of such embodiments, a DCTA molecule is produced by conjugating the polymer poly(1-lysine hydrobromide) 40,000-60,000 kD to lactoperoxidase (active moiety) and goat anti-mouse IgG antibody (targeting moiety), using sulfosuccinimidyl-4-(N-saleimidomethyl)cyclohexane-1-carboxylate (S-SMCC) as a simple linker and N-succinimidyl S-acetylthioacetate (SATA) as a modifier (see FIG. 1; further discussed in Example 1, see also U.S. Pat. No. 6,303,755 to Deo et al., regarding coupling of S-SMCC to antigen molecules).

In some embodiments, no separate linker is used and the targeting moiety is joined directly to the active moiety, with both portions of the resultant molecule retaining independent function. In such embodiments, the DCTA molecule may be synthesized using chemical methods to directly link the active and targeting moieties by chemical bond.

In specific embodiments, the linker is a complex linker, which comprises for instances a polymer or other domain, linked to the targeting and active moieties through simple linkers.

Alternatively, where the DCTA molecule is a protein, specific embodiments can be synthesized as a single expressed nucleic acid using recombinant DNA techniques such as those provided in Sambrook et al. (In Molecular Cloning: A Laboratory Manual, Cold Spring Harbor, N.Y., 1989), and Ausubel et al. (In Current Protocols in Molecular Biology, Greene Publ. Assoc. and Wiley-Intersciences, 1992). Production of such molecules is described more fully below.

In some embodiments, the DCTA molecule is synthesized using peptide linkages to assemble the component moieties, for instance as described in U.S. Pat. No. 6,307,018. In such embodiments, peptide portions of the eventual DCTA molecule are generated, then ligated together to form a native peptide linkage through intermediate steps.

### Selection of an Active Moiety

A wide variety of detection and/or isolation/separation methods are suitable for use with DCT analysis, and include but are not limited to immunohistochemistry, autoradiography, scintillation counting, mass spectrometry, affinity column chromatography, and ELISA. The choice of active moiety for inclusion in a DCTA molecule likely will be influenced by the method(s) of detection available to the user.

In certain embodiments, DCTA employs an active moiety that mediates a biochemical reaction, for instance a labeling or amplifying reaction, within the targeted cells. The active moiety in some embodiments comprises a protein, for instance a functional enzyme (e.g., reverse transcriptase, a DNA polymerase, or lactoperoxidase). Optionally, the products identified and/or amplified (e.g., through in vivo activity of a polymerase or reverse transcriptase) within the target cells can be “tagged” for subsequent analysis, identification and/or isolation (separation from its environment) by incorporation of a label or purification or separation assistive component (e.g., a radioactively labeled nucleotide, a fluorophore, a chromophore, or an epitope tag or other specific binding partner).

In yet other embodiments, the analysis is facilitated by harnessing existing machinery within targeted cells to produce products (e.g., mRNA, cDNA) that reflect an ongoing process within the target cell, in effect providing a “snapshot” (which is optionally amplified prior to or during analysis) of a molecular profile of the targeted cells. By way of example, a DCTA molecule has a targeting moiety that specific to a tumor marker (e.g., Cyclin D1/D2/D3 antibodies, Melanoma Associated Antigen) fused to an active moiety that enables amplification of cell-specific molecules within the targeted tumor cells. In one such type of DCTA molecule, the active moiety is a reverse transcriptase, which transcribes first-strand cDNA in the target cell using one or more exogenously supplied reactants. Methods of exogenously supplying such components (e.g., Na+, ATP, one or more of the four nucleotide triphosphate deoxynucleotides, and Mg²⁺) are known and have been described, for instance, by Berger and Johnson (Biochim. Biophys. Acta 425: 1-17, 1976).

Including a labeled nucleotide triphosphate, such as dCTP³², in the reaction mixture can enhance detection in embodiments that involve duplication or amplification of nucleic acids. Furthermore, products can be amplified using in vitro nucleic acid amplification techniques (e.g., PCR) to incorporate dCTP³² or another labeled nucleotide. Products labeled in this manner can be subsequently isolated, purified, and/or analyzed, such as through DNA sequencing reactions.

In one specific embodiment, the active moiety comprises a polymerase that permits in vitro amplification, rather than simply duplication. Such polymerases are known; by way of example, U.S. Pat. No. 6,033,881 provides descriptions of transcription-free isothermal methods of amplification.

B. Specific Examples of DCTA Molecules and DCT Analysis Methods

By way of example, an antibody-enzyme DCTA molecule is provided, wherein the activity of the antibody (the “targeting” moiety) is used to localize the DCTA molecule to target cells as described above. Once localized to the target cells, the enzyme moiety (“active” moiety) catalyzes a biochemical reaction within the target cells that serves (in this embodiment) to amplify the target or a detectable signal from the target within the targeted cells. This process can be performed as a means of detecting whether components of interest (e.g., proteins, mRNA, etc.) exist, or in what absolute or relative amount, in the target cells. It can also be a method by which existing components are labeled for subsequent analysis.

In some embodiments, one or more of the components necessary to support the reaction are externally provided (e.g., exogenously supplied through application to the analysis sample). In certain embodiments, at least one of the provided components is labeled (e.g., by addition of a fluorophore or radiolabel or detectable tag or separation assistive component) prior to its addition to the target sample to be analyzed.

In some embodiments, the products of the reaction of the active moiety can be isolated and analyzed using various techniques, to characterize the molecular profile of the targeted cell populations. By way of example, certain DCTA molecules have an active moiety that comprises a lactoperoxidase enzyme. Following targeting to within a sample, the lactoperoxidase is activated to iodinate tryptophan and tyrosine residues within the cells, and ¹²⁵I is provided so that the iodinated proteins are labeled. Subsequently, the labeled components are analyzed by resolving the proteins using SDS-PAGE and quantification with a gel scintillation counter. Alternatively, the gels are exposed to film and the signal is quantified by measuring signal intensities.

In another example, an antibody-enzyme DCTA molecule is provided, and linked to a chromatography column. The activity of the antibody (targeting moiety) is used to bind the DCTA molecule to target cells in a sample (e.g., cell suspension mixture) that is passed over the column. Following targeting, the enzyme (active moiety) is used to label the proteins that are bound to the targeting moiety antibody. In some embodiments, the bound molecules are eluted from the column for subsequent analysis. In certain embodiments, at least one of the provided components is labeled (e.g., by addition of a fluorophore or radiolabel) prior to addition to the chromatography column.

In yet another example, an antibody-enzyme DCTA molecule is provided, and linked to a support medium (e.g., a plate containing multiple wells, such as an ELISA plate). The activity of the antibody (targeting moiety) is used to bind the DCTA molecule to target cells in a mixture of proteins applied to the support medium. Following targeting, the enzyme (active moiety) is activated such that it generates a signal when target cells or cell components are present. In some embodiments, the active moiety is a chromogen or substrate (e.g., ABTS/H₂O₂), which turns color or emits a detectable signal when the enzyme (e.g., horseradish peroxidase) is activated.

In some embodiments, a label is added to the DCTA molecule itself prior to targeting it to the sample, e.g., a fluorophore, a radioisotope, or a luciferase or like detectable molecule. The label can either be directly or indirectly attached to the DCTA molecule. For instance, a fluorophore may be attached indirectly to the DCTA molecule by a linker molecule.

In embodiments in which the DCTA molecule is an engineered fusion protein synthesized from fusion nucleic acid, the DCTA molecule can be labeled by including a sequence encoding a fluorophore (e.g., luciferase, GFP, or another fluorescent protein). By way of example, U.S. Pat. No. 6,232,107 teaches the use of sequences encoding fluorophores and luciferases in plasmid vectors, and methods of synthesizing and purifying the encoded nucleic acids.

Following targeting of a DCTA molecule containing a label, the label can be used to visualize the targeted cells to provide a means of visualizing the targeted cells via microscopy or other means.

C. Localization within Target Cells.

### Sample Selection and Preparation

DCT analysis methods are useful in analyzing a variety of samples, including biopsy material, tissue sections, cell culture preparations, cytology preparations, cells in vitro, and cells in vivo.

In some embodiments, suitable cells for targeting include a suspension of cells in a solution containing protease inhibitors, such as a selection of cells harvested from a cell culture preparation. Optionally, such suspended cells can be immobilized, e.g., by gelling the solution with a polymer.

In certain embodiments, target molecules (e.g., proteins, carbohydrates, etc.) are exposed for targeting in the sample without the need for other manipulation to enhance interaction of the DCTA molecule, such as treatment with a perforating buffer (e.g., saponin) or solubilization of a binding medium (e.g., cyanogen bromide solubilization of transferrin polypeptides and glycopolypeptides from formalin-fixed, paraffin wax-embedded tissue sections, see Brooks et al., Histochem J. 30(8): 609-15, 1998; de-waxing of ethanol-fixed, paraffin-embedded tissue sections in xylenes, followed by hydration and equilibration in Tris-buffered saline with Tween-20, etc.). Examples of such samples in which exposed molecules can be found include cytology preparations or freshly ethanol-, methanol-, or acetone-fixed tissue sections.

In some embodiments, samples are prepared for DCTA using a fixative (e.g., methanol) that permeabilizes the sample to enhance the ability of an applied DCTA molecule to interact with the tissue sample, such as to allow the DCTA molecule to penetrate below the surface of a tissue section. In certain embodiments, pretreatment of a prepared cell or tissue sample is performed (e.g., exposure to proteinase K buffer, incubation with saponin detergent, polylysine, polyarginine, poly (lysine-arginine) or similar polypeptides, such as polycationic dendrimers) to enhance the ability of biomolecules (e.g., the DCTA molecule) to interact with the sample (see Masuda et al., Nucleic Acids Res. 27(22): 44364443, 1999). In some embodiments, including those in which the DCTA molecule interacts with surface molecules only, no treatment is necessary.

### Application of DCTA Molecule

Once constructed, the DCTA molecule is applied to a tissue section or preparation of cells (e.g., a tissue section, cell culture preparation, cytology preparation, or cells in vivo), and allowed to localize based on its binding affinity. In some embodiments, the DCTA molecule will be added to an in vitro sample consisting of solubilized cells. In other embodiments, the DCTA molecule is applied by solubilizing the molecule in a buffer (e.g., 50 mM Tris-HCl, pH 7.5, 1% Triton X-100) and applying the buffer to the sample, such as by direct application to a tissue section. The DCTA molecule solution may optionally include an enzyme inhibitor, such as an RNase inhibitor, a DNase inhibitor, a protease inhibitor, and mixtures thereof. In some embodiments, the DCTA molecule is applied by automated means, such as by an automated immunostainer during an ELISA procedure (e.g., a Dako instrument, Dako Corporation, Carpenteria, Calif.).

In some embodiments, the samples are washed during DCTA (e.g., application of a wash buffer to a sample on a slide, suspension of a sample pellet with a wash buffer, rapidly dipping a slide bearing the sample in and out of a wash solution, and washing by automated means). Useful solutions for washing the tissue sample include, but are not limited to, phosphate-buffered saline (PBS), distilled water, diethylpyrocarbonate (DEPC) treated water, Tris-buffered saline (TBS), ethanol-water solutions, RNAsecure™ (Ambion, Austin, Tex.) and mixtures thereof. The wash solution may also include a surfactant, such as a nonionic detergent, for example Tween 20, 40, 60, 80, or 100.

### Detection of Generated Signal

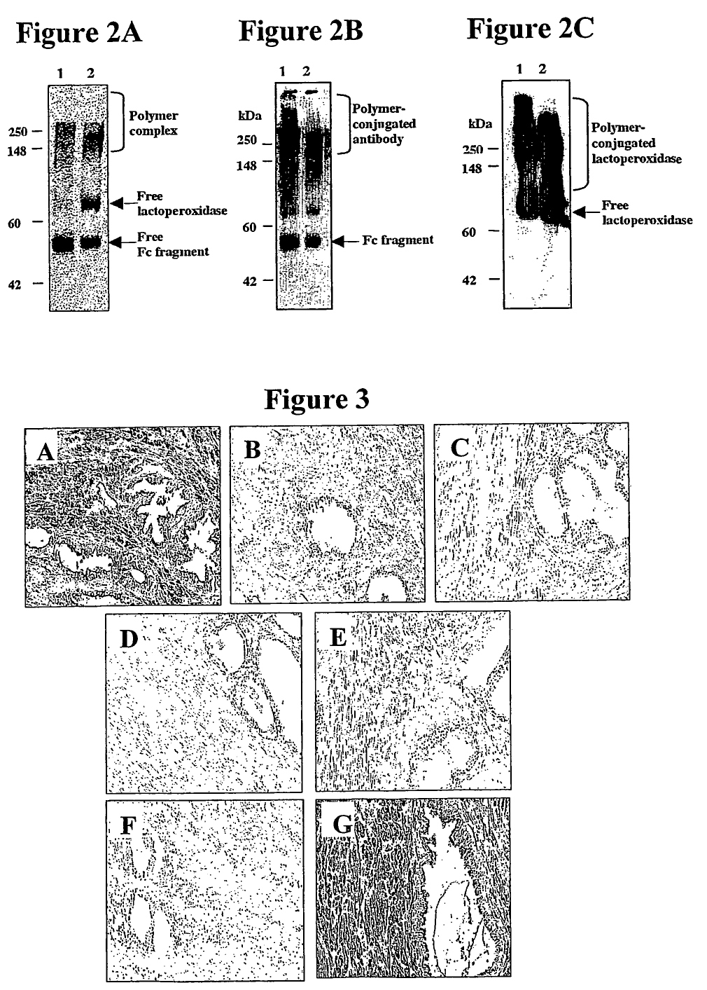

Localization of the DCTA molecule within the sample optionally can be confirmed by detecting the targeted molecules in the sample. The method of detection may be influenced by the type of DCTA molecule chosen, for instance by whether or not the molecule includes a detectable label. By way of example, a targeting moiety consisting of an antibody (e.g., goat anti-mouse IgG) can be detected through application of a secondary antibody designed to detect the first antibody (e.g., HRP-conjugated rabbit anti-goat IgG, or rabbit anti-lactoperoxidase IgG followed by HRP-conjugated mouse anti-rabbit IgG, for instance). In such embodiments, detection is achieved through extraction of the proteins from the sample and separation of the proteins by size (e.g., SDS-PAGE), followed by visualization using an appropriate method (e.g., blot staining with Ponceau S, autoradiography, etc.) (see FIG. 2, Example 1).

In embodiments in which a fluorophore or luciferase molecule is used to label the DCTA molecule, these molecules can be detected after they are targeted within a sample, and their localization visualized using appropriate methods (e.g., using microscopy and photography, luminography, etc.)

D. Active Moiety Function

Following localization, the active moiety facilitates the analysis of the target cells or cellular components by generating a detectable signal or providing a distinguishing characteristic to the target component or the cell in which it occurs, based on the activity of the active moiety.

The active moiety acts upon all suitable reactants within reach (e.g., a lactoperoxidase active moiety iodinates all tryptophan and tyrosine residues within its range). Based on the specificity of the DCTA targeting, only molecules from the targeted cells are acted upon, so only molecules from these cells will be detected/isolated/further analyzed. The biomolecules not in a targeted cell or region are “invisible” (in that they are, in different embodiments, not labeled or occur at too low a relative level to be detected, compared to the biomolecules at the target), and hence are no a significant influence in subsequent analysis.

In some embodiments, a DCTA molecule may be synthesized to include a long linker, which would broaden the radius of activity for the active moiety (see “Selection of Linker Molecules” above).

The following descriptions correspond to specific examples of active moieties and their associated function. Other active moieties are contemplated as described herein.

### Isotopic Labeling

In some embodiments, the active moiety labels existing elements within the cell, such as proteins and other biomolecules. By way of example, a lactoperoxidase active moiety is used in specific embodiments to label proteins in the target cells with exogenously applied ¹²⁵I. Subsequently, the ¹²⁵I-labeled cells/molecules can be analyzed to determine a molecular profile of the labeled cells, for instance through visualization of the labeled products (e.g., using autoradiography or measurement with a gamma counter).

In some embodiments, the active moiety is used to label existing elements within the cells with isotopically different forms of a reagent. Such methods can be used to illustrate existing differences between two types of samples (e.g., tumor and non-tumor cells), or between two different targets within the same sample (e.g., seritonergic and dopaminergic receptors in a brain tissue slice, mutant and non-mutant variants of a target receptor using a targeting moiety directed to the known sequences, intra- and extra-cellular domains of a transmembrane protein, transcripts produced after stimulus with a pharmaceutical or ionizing radiation), where the two samples/targets are labeled with different isotopic reagents and the resultant molecular profiles compared.

Applicable labeling reagents for use in such methods include isotope-coded affinity tags (ICAT), which have a heavy form (containing deuterium), and a light form (containing hydrogen), or other pairs of isotopes, such as heavy and light forms of nitrogen (see Gygi et al., Nat. Biotech. 17: 994-999, 1999). Following incorporation of the isotopic reagent, the samples are analyzed and compared using differences between the types of reagents, for instance using mass spectrometry. By way of example, a mass spectrometer uses a dual mode to measure the relative signal intensities for pairs of peptide ions of identical sequence that are tagged with the isotopically heavy or light forms of the reagent, and that therefore differ in mass by the mass differential encoded within the reagent In such embodiments, DCT analysis can be used to profile different types of cells (e.g., tumor and non-tumor cells) by assigning one type of reagent to each cell, and comparing molecular profiles of cells of each type (e.g., levels of protein, mRNA, etc.).

### Production/Amplification/Labeling of Nucleic Acids

In another embodiment, the active moiety employs existing cell machinery to synthesize an entirely new product that reflects a characteristic of the target cells. By way of example, a DCTA molecule comprising reverse transcriptase as its active moiety generates a detectable signal by transcribing first-strand cDNA from mRNA in the target cell using exogenously supplied nucleotide triphosphate molecules. Through use of a labeled nucleotide triphosphate, such as dCTP³², a user can quantitatively compare the components of a cell (e.g., tumor versus non-tumor cells), to garner a “snapshot” of those nucleic acid molecules being actively expressed within targeted cells.

### In Situ Generation of Products for Optional Separation

In some embodiments, the active moiety produces a product (e.g., where reverse transcriptase active moiety generates transcripts, or where lactoperoxidase labels proteins with a radioisotope), these products optionally can be extracted and/or purified and subsequently analyzed (e.g., isolation of a target protein from gel using incorporated radionucleotides as means of locating the protein on a gel). In some embodiments, it will not be necessary to extract and/or purify the products from the sample, as the user is only interested in obtaining information regarding the targeted cells or sub-cellular components within the sample (e.g., existence or distribution of a cell receptor or of a gene transcript, such as in a comparison of cell receptors or transcripts among cancerous and non-cancerous samples; see Example 4).

In some embodiments, DCTA molecules may be made to have a DNA probe as the targeting moiety, and an active moiety that labels mRNA transcripts in situ, using a method allowing for subsequent visualisation (e.g., addition of a fluorophore or luciferase). Following targeting and labeling, the labeled sequences are visualized by microscopy and photography or other applicable means. Alternatively, the labeled sequences may be separated by scraping the sample into a tube and performing further analysis (e.g., sequencing or further amplification using polymerase chain reaction and subsequent visualization on a gel, or in an array, etc.).

V. Production of Protein DCTA Molecules Using Recombinant DNA Techniques

Specific DCTA molecules of the disclosure that are proteins can be synthesized using recombinant DNA techniques, such as those provided in Sambrook et al. (In Molecular Cloning: A Laboratory Manual, Cold Spring Harbor, N.Y., 1989), and Ausubel et al. (In Current Protocols in Molecular Biology, Greene Publ. Assoc. and Wiley-Intersciences, 1992). The following method of expression of DCTA molecules is provided merely by way of example. One skilled in the art will understand that there are myriad ways to express a recombinant protein such that it can be subsequently purified. See, for instance, U.S. Pat. No. 5,089,400 (“Polypeptides and process for the production thereof”).

In general, an expression vector carrying the nucleic acid sequence that encodes the desired protein will be transformed into a microorganism for expression. Such microorganisms can be prokaryotic (bacteria) or eukaryotic (e.g., yeast). One appropriate species of bacteria is Escherichia coli (E. coli), which has been used extensively as a laboratory experimental expression system. A eukaryotic expression system will be preferred where the protein of interest requires eukaryote-specific post-translational modifications such as glycosylation.

The expression vector can include a sequence encoding the targeting moiety, positioned in such a way as to be fused to the coding sequence of the active moiety. This allows the DCTA molecule to be targeted to specific locations by the targeting moiety while carrying the active moiety. In a prokaryotic expression system, a signal sequence can be used to secrete the newly synthesized fusion protein. In a eukaryotic expression system, the targeting moiety would specify targeting of the DCTA molecule to one or more specific cells or sub-cellular compartments, depending on which moiety is chosen. Appropriate targeting moieties include an antibody directed to a specific cell receptor, such as a seritonergic receptor in brain tissue or prostate membrane specific antigen (PMSA) (see Renneberg et al., Prostate 46(3): 173-83, 2001).

Vectors suitable for stable transformation of bacterial cells are well known. Typically, such vectors include a multiple-cloning site suitable for inserting a cloned nucleic acid molecule, such that it will be under the transcriptional control of 5′ and 3′ regulatory sequences. In addition, transformation vectors include one or more selectable markers; for bacterial transformation this is often an antibiotic resistance gene. Such transformation vectors typically also contain a promoter regulatory region (e.g., a regulatory region controlling inducible or constitutive expression), a transcription initiation start site, a ribosome binding site, an RNA processing signal, and a transcription termination site, each functionally arranged in relation to the multiple-cloning site. For production of large amounts of recombinant proteins, an inducible promoter is preferred. This permits selective production of the recombinant protein, and allows both higher levels of production than constitutive promoters, and enables the production of recombinant proteins that may be toxic to the expressing cell if expressed constitutively.

In addition to these general guidelines, protein expression/purification kits have been produced commercially. See, for instance, the QIAexpress™ expression system from QIAGEN (Chatsworth, Calif.) and various expression systems provided by INVITROGEN (Carlsbad, Calif.). Depending on the details provided by the manufactures, such kits can be used for production and purification of the disclosed DCTA molecules (see IX. “Kits for Direct Cell Target Analysis”).

VI. Additional Applications and Molecules

A. Standardized Direct Cell Target Analysis Molecules

In addition to the target-specific DCTA molecules described herein, also provided are DCTA molecules wherein the targeting moiety comprises a secondary antibody binding domain or other generalizable or standardized targeting moiety for use in multiple targeting applications. In examples where the binding moiety comprises a “standardized” binding domain, the DCTA molecule can be used to detect multiple different targets. A generalizable or standardized targeting moiety is one that can detect multiple different targets, for instance, through an adapter molecule such as a primary antibody that is used to effect operative interaction compatibility between the standardized DCTA molecule and the specific target. A representative standardized targeting moiety is a “secondary” antibody or antibody binding domain, such as an anti-IgG antibody, where the antibody recognizes IgG produced in a different species than that used to produce the primary antibody (which acts as the adapter molecule). By way of specific example, the secondary antibody is a mouse anti-goat IgG.

In using standardized DCTA molecules, a user selects an adapter molecule such as a primary antibody (for instance, a commercial primary antibody) directed to the specific target of interest. Specific examples include an antibody directed to p53 mutation-positive tumor cells (e.g., which recognizes an epitope specific to the p53 mutation), an antibody specific for tumor cells (e.g. which recognizes an identified tumor over-expression marker or associated protein), or an antibody that recognizes cells in a particular stage of the cell cycle (e.g., based on expression of a cell-cycle specific protein), or an antibody specific for subsets of cell types that are uniquely expressing a given protein. It is particularly contemplated that commercially available primary antibodies or laboratory-prepared primary antibodies can be used.

The primary antibody is applied to the sample, where it localizes to the target cells or cell components based on specific binding characteristics, then the standardized DCTA molecule is applied. The secondary antibody generalized targeting domain of the DCTA molecule specifically binds to the primary antibody (which could itself be viewed as the specific “targeting moiety” in this example) that is already bound to cells. Subsequently, the active moiety of the DCTA molecule is activated to act upon or within the target cells. The tissue sample is then analyzed, e.g., the targeted molecules are detected, quantified or purified using applicable techniques as described elsewhere herein.

B. Detection of Mutations

Other embodiments provide methods to detect the presence of a mutation in a gene. Such methods can be used, for instance, as a means of detecting or diagnosing cancer (e.g., detecting a mutation in the p16 gene to diagnose invasive esophageal squamous cancer, detecting a BRCA1 or BRCA2 mutation to diagnose ovarian cancer, detecting a mutation on chromosome 8p to diagnose prostate cancer, etc). In specific examples of these methods, the targeting moiety comprises a DNA probe that anneals to the region flanking the putative mutation on the target gene, and the active moiety is a polymerase that amplifies the mutated region of the gene. Following polymerization, the entire tissue sample is scraped into a tube and analyzed (e.g., sequenced or further amplified using polymerase chain reaction and subsequent visualization on a gel). Amplified sequences can be identified based on their abundance or by location of a tag (such as a fluorophore or radioisotope) incorporated during the amplification reaction. Detection of a mutation with these methods can serve as a biochemical marker that is indicative of the disease.

C. Generation of Antibody for Use in Direct Cell Target Analysis

In some embodiments, a user may desire to generate an antibody for use in DCT analysis. Methods of generation of antibodies are well known. For example, an antigen specific to a type of cell within a tissue sample may be produced by injecting the antigen into a host animal, repeatedly, over a period of time. If the desired antigen is a small molecule, it may be conjugated to a large protein, such as Bovine Serum Albumin, before it is administered to the host animal. Suitable host animals for this purpose include mammals such as rabbits, horses, goats, guinea pigs, rats, cows, sheep, etc. The serum is collected from the host animal and the antibody is precipitated with a neutral salt solution and purified by dialysis and column chromatography. The resulting polyclonal antibody, is actually a multiplicity of antibodies that selectively complex with the antigen.

Alternatively, lymphocytes are isolated from the host animal and fused to myeloma cells. The resulting hybridoma cells are selected based on their production of antibodies toward the antigen, cloned, and cultured. The antibody is then isolated from the culture medium. The antibody isolated from the clones of a particular hybridoma is termed a monoclonal antibody. Finally, either a monoclonal or polyclonal antibody preparation is treated with a reactive fluorescent moiety to produce fluorescent conjugates of antibodies, which may be purified, for example, by size exclusion chromatography. Commercial kits for producing fluorescent conjugates of antibodies and other proteins are available (for example, the Alexa Fluor Protein Labeling Kit from Molecular Probes, Inc., Eugene, Oreg.).

Following purification and testing of the generated antibody, the antibody may be used as a targeting molecule in a DCTA molecule (see Example 1 for representative synthesis conditions).

D. Detection of Injured Kidney Tissue

In injured kidney tissue, expression of the Tamm-Horsfall protein is known to decrease following renal ischemia; detection of ischemic thick ascending limbs with fluorescent antibodies against the Tamm-Horsfall protein is difficult. However, the amount of Na—K-2Cl co-transporter does not decrease following ischemia (see Fernandez-Llama et al., J. Am. Soc. Mephrol., 10: 1658-1668, 1999 and Kwon et al., Am. J. Physiol. Renal Physiol., 278: F925-F939, 2000). Thus, antibodies directed toward the co-transporter protein are useful for identifying ischemic thick ascending limbs.

In a specific DCTA embodiment, a DCTA molecule is made that has an antibody directed to the Na—K-2Cl co-transporter protein, and an active moiety that labels the protein in situ. Following targeting and labeling, the labeled proteins are visualized by microcopy and photography or other applicable means. Alternatively, the entire tissue sample may be scraped into a tube and analyzed (e.g., sequenced or further amplified using polymerase chain reaction and subsequent visualization on a gel).

VII. Automation of Direct Cell Target Analysis Procedures

DCTA can be automated to increase the efficiency of producing and analyzing samples. Automation also can provide the additional benefits of reduced operator error, increased consistency and the ability to process a greater number of samples.

In one embodiment, multiple tissue section slides are fixed to individual supporting media (e.g., glass slides), and the samples are subjected to automated ELISA (e.g., Trinity Biotech ELISA Processor, Trinity Biotech PLC, Co Wicklow, Ireland) using a DCTA molecule, for instance where the DCTA molecule is an antibody-enzyme enzyme fusion. After processing of the ELISA using an automated immunostainer (e.g., a Dako instrument, Dako Corporation, Carpenteria, Calif.), the activity of the active moiety is automatically triggered, for instance by automated addition of necessary reagent(s). The components of interest then can be automatically separated from the support medium environment, and the components of interest can be analyzed subsequently. In examples of such embodiments, each slide contains several hundred thousand to a million cells of interest, and DCTA is completed in a few hours, as opposed to the greater than one week or more of operator time necessary for microdissection alone using traditional laser capture microdissection techniques.

In another automated embodiment, the DCTA molecule consists of a cell-specific antibody targeting moiety and a DNA polymerase active moiety. The DCTA molecule is applied by automated means, such as an automated ELISA (e.g., Trinity Biotech ELISA Processor, Trinity Biotech PLC, Co Wicklow, Ireland), and components of the polymerization reaction are exogenously supplied using methods such as those described by Berger and Johnson (Biochim. Biophys. Acta 425(1): 1-17, 1976). A label can be incorporated into products of the reaction to facilitate subsequent analysis (e.g., incorporation of radiolabeled nucleotide molecules enables identification of the synthesized products by autoradiography or scintillation gel counting). Following activation of the DCTA molecule, the products of the individual DCT analysis reactions are automatically harvested into an appropriate solution (e.g., buffer) for storage and subsequent analysis (e.g., visualization by autoradiography, sequencing, etc.).

VIII. Genomics and Proteomics

The disclosure also includes methods that combine DCT analysis of pure populations of cells and cell components with other technologies, such as high-throughput genomics, to identify molecular characteristics, such as structural changes in genes or proteins, copy number or expression alterations of genes, with disease prognosis or therapy outcome, to identify novel targets for gene prevention, early diagnosis, disease classification, or prognosis, and to identify therapeutic agents. Such high-throughput technologies include cDNA and genomic sequencing, serial analysis of gene expression (SAGE), representational difference analysis (RDA), differential display and related PCR-based technologies, hybridization-based sequencing, subtractive cDNA or genomic hybridizations, cDNA arrays, CGH arrays, electrophoretic, mass spectrometric, or other separation and identification methods (including SELDI fingerprinting) for DNA or proteins, yeast two-hybrid technology or related techniques of molecular biology.

A particular example of high throughput proteomic technique that may be combined with the methods of the present disclosure is SELDI protein fingerprinting. SELDI analysis of proteins from samples analyzed according to the DCT analysis methods of the disclosure may be used, for example, to assess changes in protein expression occurring during tumor progression following analysis. SELDI analysis of pure populations of cells and tissue structures obtained by DCTA will provide a more complete picture of cell level proteomics that includes proteins with cell surface receptors, for instance. Such information will aid in the elucidation of the fundamental mechanisms underlying disease and identification of markers that may be utilized for diagnostic purposes. Such analyses are not however restricted to a particular disease state and may also be utilized to elucidate mechanisms of tissue damage and repair in response to injury, chemical, physical, or otherwise.

Pure cell and tissue structure samples analyzed according to the DCT analysis methods of this disclosure may also be used in combination with array techniques and can provide information about the frequency of a multitude of genetic alterations or gene expression patterns (including normal gene expression patterns) in a variety of tissue types (such as different types of tumors), and in tissue of a particular histological type (such as a tumor of a specific type, such as intraductal breast cancer), as well as the tissue distribution of molecular markers tested. Differential gene expression, which can be detected by varying levels of proteins or RNA detected by this technique (e.g., by DCTA, then semi-quantitative RT-PCR), can then be used for diagnostic or therapeutic purposes. For example, overexpression or underexpression of particular proteins can be associated with particularly benign or malignant tumors, to provide prognostic information about the likely clinical course of a tumor (and decide whether aggressive chemotherapy must be undertaken). Similarly, information about differential protein expression in particular types of disease (such as tumors of a particular type) can be used to target treatment. Hence if upregulation of a protein is found in a particular type of tumor cell, therapies aimed at disruption of that upregulation can be administered. The use of the DCT analysis methods disclosed herein, for both diagnostic and therapeutic purposes, is therefore included.

IX. Kits for Direct Cell Target Analysis

DCTA molecules described herein, including the standardized or general DCTA molecules, are ideally suited for use in a kit for making the DCTA molecule and for performing DCT analysis methods.

Kits includes a carrier means, such as a box, a bag, or plastic carton. In one embodiment the carrier contains one or more containers, for instance vials, tubes, and the like, that include a pre-made DCTA molecule, or with precursors that may be combined to create a DCTA molecule. In some embodiments, the carrier includes a container with reagents for use with the DCTA molecule, such as a buffer, or a vehicle for the introduction of the DCTA molecule to the tissue sample.

Instructions can be provided to detail the use of the components of the kit, such as written instructions, video presentations, or instructions in a format that can be opened on a computer (e.g. a diskette or CD-ROM disk). These instructions indicate, for example, how to make a DCTA molecule using the kit, how to use a DCTA molecule to isolate and/or analyze cells or components of cells of interest, or how to use a DCTA molecule to generate molecular profiles of tissue samples.

Specific provided kits contain a standardized or general DCTA molecule, wherein the targeting moiety is a secondary antibody that has affinity for a number of different primary antibodies (for instance, an anti-IgG antibody reactive to IgG produced from a different species). Using such kits, the user can select a primary antibody based upon the desired target population, and use the general DCTA molecule as a means of secondary detection and subsequent analysis.

The amount of each DCTA molecule supplied in the kit can be any appropriate amount, depending for instance on the market to which the product is directed. For instance, if the kit is adapted for research or clinical use, the amount of each DCTA molecule provided would likely be an amount sufficient to screen several tissue samples. The substance(s) can be provided suspended in an aqueous solution or as a freeze-dried or lyophilized powder, for instance.

Those of ordinary skill in the art know the amount of each moiety in a DCTA molecule that is appropriate for use in a single detection reaction. General guidelines may for instance be found for the use of antibodies, DNA probes, and receptor binding reactions in Innis et al. (PCR Protocols, A Guide to Methods and Applications, Academic Press, Inc., San Diego, Calif., 1990), Sambrook et al. (n Molecular Cloning: A Laboratory Manual, Cold Spring Harbor, N.Y., 1989), and/or Ausubel et al. (In Current Protocols in Molecular Biology, Greene Publ. Assoc. and Wiley-Intersciences, 1992).

Kits may additionally include one or more buffers for use during detection procedures. For instance, such buffers may include a low stringency, a high stringency wash, and/or a stripping solution. These buffers may be provided in bulk, where each container of buffer is large enough to hold sufficient buffer for several probing or washing or stripping procedures. Alternatively, the buffers can be provided in pre-measured aliquots, which can be tailored to the size and style of tissue targeting substance included in the kit.

Furthermore, kits may include positive and negative controls (e.g., tissue sections of known molecular profile, or control DCTA molecules with a known activity, such as DCTA molecules targeted to housekeeping genes) for use in confirming that the DCTA molecule is effective as applied. Kits may also include one or more reagents necessary to support activity of the active moiety of a DCTA molecule (e.g., nucleotide triphosphates, enzyme catalysts, secondary detection antibodies, tissue section stains, etc.) or general components useful in the reaction (e.g., slides and coverslips, tubes, etc.).

Without further elaboration, it is believed that one skilled in the art can, using this description, utilize the present disclosure to its fullest extent The following examples are illustrative only, and not limiting of the remainder of the disclosure in any way whatsoever.

### EXAMPLES

### Example 1

### Construction and Characterization of Direct Cell Target Analysis Molecules

This example provides representative methods for constructing and confirming the functionality of a DCTA molecule using mammalian tissue samples.

Synthesis of a DCTA Molecule.

All reagents were prepared in 0.1 M sodium phosphate buffer, which included 0.15 M NaCl, pH 7.2. 500 mM of sulfosuccinimidyl-4-(N-maleimidomethyl)cyclohexane-1-carboxylate (Sulfo-SMCC, Sigma, St. Louis, Mo.) was added to 50 mM of L-lyso-hydrobromide polymer (Polysciences Inc., Warrington, Pa.) at a 200:1 molar ratio of sulfu-SMCC:polymer (see FIG. 1 for schematic of synthesis reaction) The reaction was incubated for 30 minutes at room temperature and immediately purified in a 2-ml desalting column using 0.1 M sodium phosphate, 0.15 M NaCl, pH 7.2 as the elution buffer. The fractions containing the maleimide-activated polymer were identified by measuring the protein content at 280 nm (see FIG. 7). The fractions selected for further purification were those having the earliest and highest peaks.

Subsequently, goat anti-mouse IgG antibodies (24 μM) (Sigma, St. Louis, Mo.) and lactoperoxidase (200 μM) were modified by the attachment of the modifier S-acetylthioglycolic acid N-hydroxysuccinimide ester (SATA, Sigma, St. Louis, Mo.) at a 50 molar excess of SATA. The SATA-linked lactoperoxidase and antibodies were activated by incubating the samples with hydoxylamine (50 mM) for two hours at room temperature. The reactions were immediately purified using a desalting columns (Pierce, Rockford, Ill.) and 50 mM sodium phosphate, 10 mM EDTA, pH 7.5 as the elution buffer. The fractions were collected and the protein content was measured at 280 nm to identify fractions containing maleimide-activated polymer. Immediately, the thiolated lactoperoxidase and antibodies were mixed with the maleimide-activated polymer at a molar ratio of 10 (each) to 1. The reactions were incubated for 25 minutes at room temperature, then at 37° C. for 40 minutes. The reaction was centrifuged for 10 minutes at 14,000×g. The supernatant (1500 μl) containing soluble polymer (“polymer supernatnat”), was separated form the pellet containing insoluble polymer (“polymer pellet”). 400 μL of 0.1 M sodium phosphate, 0.15 M NaCl, pH 7.2 were added to the insoluble polymer, which was resuspended by vigorous vortexing and by grinding with a pestle. Both polymer pellet and polymer supernatant were aliquoted and stored at −20° C. The molecule as synthesized constituted one example of a “DCTA molecule” of the disclosure.

Confirmation of Polymer Linkage to Antibodies and Lactoperoxidase Molecules.

To confirm that the targeting and active moieties were successfully linked to the polymer molecule, 20 μl of each polymer pellet (lane 1) and polymer supernatant (lane 2) were resolved on a 4-20% SDS-PAGE gel, then transferred to a nitrocellulose membrane (InVitrogen, Carlsbad, Calif.). Proteins remaining in the gel were then stained with GelCode Blue Stain Reagent (Pierce, Rockford, Ill.) (see FIG. 2A). Transferred proteins were immunoblotted with either horseradish peroxidase (HRP)-conjugated rabbit anti-goat IgG antibodies (Pierce, Rockford, Ill.) (see FIG. 2B) or rabbit anti-lactoperoxidase antibodies followed by HRP-conjugated mouse anti-rabbit IgG antibodies (Pierce, Rockford, Ill.) (see FIG. 2C). It was visually confirmed that high-molecular weight bands representing the polymer linked to the antibody and lactoperoxidase molecule were present in both the polymer pellet and polymer supernatant samples, in addition to free molecules.

Confirmation of Functionality of Targeting Antibody Moiety of DCTA Molecule.

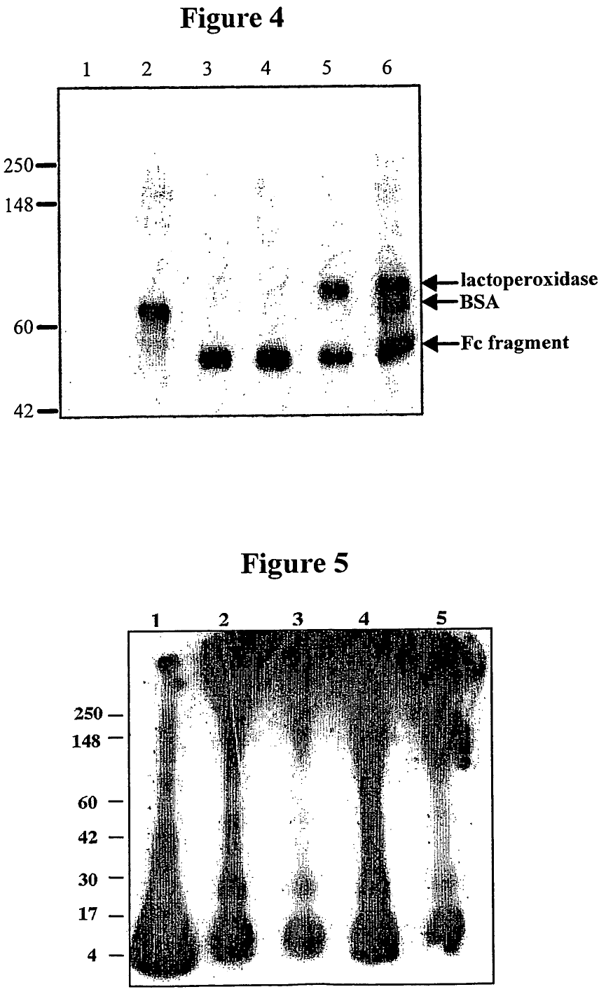

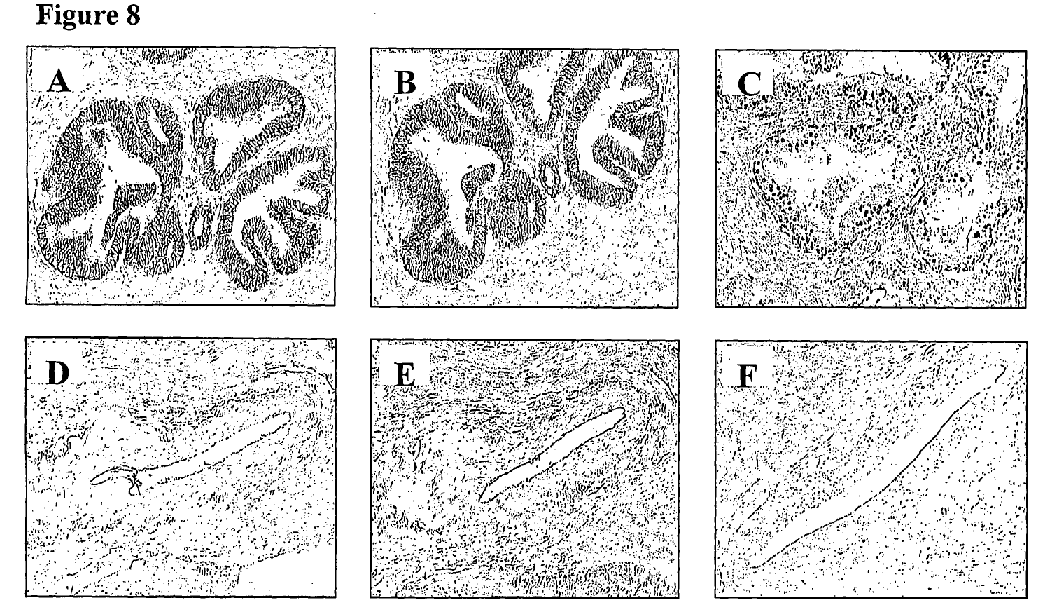

To confirm that the antibodies as linked to the DCTA molecule (“polymer”) were functional, the ability of the antibodies to bind targeted molecules in cells was investigated (see FIG. 4A-G and FIG. 8A-F). Immunohistochemistry was carried out using the EnVision+ system (Dako Corporation, Carpenteria, Calif.). Briefly, ethanol-fixed, paraffin-embedded prostate tissue sections placed on glass slides were de-waxed in xylene (Mallinckrodt, Hazelwood, Mo.), hydrated, and equilibrated in Tris-buffered saline with Tween-20 (0.05%). The following antibody binding reactions were then performed according to the manufacturer's recommendations:
4A. Mouse anti-tropomyosin IgG (Biomeda, Foster City, Calif.) (1:1000), followed by Dako's HRP-labeled polymer conjugated to goat anti-mouse IgG (Dako Corp., Carpenteria, Calif.), used in the dilution provided by the manufacturer to test for the presence of tropomyosin in prostate cell populations. (see FIG. 3A),
4B. Rabbit anti-lactoperoxidase antibodies (Pierce, Rockford, Ill.) (1:1000) followed by Dako's HRP-conjugated to goat anti-rabbit IgG (Dako Corp., Carpenteria, Calif.) used in the dilution provided by the manufacturer to test for internal expression of lactoperoxidase in prostate cell populations. (see FIG. 3B),
4C. Mouse anti-tropomyosin IgG (1:1000), followed by Dako's HRP labeled polymer conjugated to goat anti-rabbit IgG antibodies (Dako Corp., Carpenteria, Calif.) used in the dilution provided by the manufacturer to confirm the inability of the anti-rabbits IgG antibody to bind non-specifically to the mouse anti-tropomyosin IgG (see FIG. 3C),
4D. Polymer supernatant (1:10) followed by Dako's HRP-conjugated to goat anti-rabbit IgG (Dako Corp., Carpenteria, Calif.) used in the dilution provided by the manufacturer to confirm the inability of the goat anti-rabbit antibodies to bind non-specifically to the polymer complex that might have bound protein molecules non-specifically (see FIG. 3D),
4E. Mouse anti-tropomyosin IgG (1:1000) followed by polymer supernatant, then Dako's HRP labeled polymer conjugated to goat anti-rabbit IgG (Dako Corp., Carpenteria, Calif.) used in the dilution provided by the manufacturer to confirm the inability of the secondary anti-rabbit antibodies to bind non-specifically to the polymer complex bound to the mouse anti-tropomyosin IgG (see FIG. 3E),
4F. Polymer supernatant (1:10) followed by rabbit anti-lactoperoxidase (1:1000), then Dako's HRP labeled polymer conjugated to goat anti-rabbit IgG (Dako Corp., Carpenteria, Calif.) used in the dilution provided by the manufacturer to confirm the inability of the polymer complex to bind non-specifically to proteins expressed by prostate cell populations (see FIG. 3F),
4G. Mouse anti-tropomyosin IgG (1:1000), followed by polymer supernatant, then rabbit anti-lactoperoxidase antibodies (1:1000), then Dako's HRP labeled polymer conjugated to goat anti-rabbit IgG (Dako Corp., Carpenteria, Calif.) used in the dilution provided by the manufacturer to detect the ability of the polymer complex to recognize the primary mouse antibodies via recognition of the polymer-linked lactoperoxidase (see FIG. 3G),
8A. Mouse anti-E-cadherin IgG (Transduction Laboratories, Lexington, Ky.) (1:200), followed by Dako's RP-labeled polymer conjugated to goat anti-mouse IgG (Dako Corp., Carpenteria, Calif.), used in the dilution provided by the manufacturer to test for the presence of E-cadherin in prostate cell populations (see FIG. 8A),
8B. Mouse anti-E-cadherin IgG (Transduction Laboratories, Lexington, Ky.) (1:200), followed by polymer supernatant, then rabbit anti-lactoperoxidase antibodies (1:1000), then Dako's HRP labeled polymer conjugated to goat anti-rabbit IgG (Dako Corp., Carpenteria, Calif.) used in the dilution provided by the manufacturer to detect the ability of the polymer complex to recognize the primary mouse antibodies via recognition of the polymer-linked lactoperoxidase (see FIG. 8B),
8C. Polymer supernatant (1:10) followed by rabbit anti-lactoperoxidase (1:1000), then Dako's HRP labeled polymer conjugated to goat anti-rabbit IgG (Dako Corp., Carpenteria, Calif.) used in the dilution provided by the manufacturer to confirm the inability of the polymer complex to bind non-specifically to proteins expressed by prostate cell populations (see FIG. 8C),
8D. Mouse anti-CD34 IgG (Immunotech, Marseilles, France) (1:200), followed by Dako's HRP-labeled polymer conjugated to goat anti-mouse IgG (Dako Corp., Carpenteria, Calif.), used in the dilution provided by the manufacturer to test for the presence of CD34 in prostate cell populations (see FIG. 8D),
8E. Mouse anti-CD34 IgG (Immunotech, Marseilles, France) (1:200), followed by polymer supernatant, then rabbit anti-lactoperoxidase antibodies (1:1000), then Dako's HRP labeled polymer conjugated to goat anti-rabbit IgG (Dako Corp., Carpenteria, Calif.) used in the dilution provided by the manufacturer to detect the ability of the polymer complex to recognize the primary mouse antibodies via recognition of the polymer-linked lactoperoxidase (see FIG. 8E), and
8F. Mouse anti-CD34 IgG (Immunotech, Marseilles, France) (1:200), followed by Dako's HRP labeled polymer conjugated to goat anti-rabbit IgG antibodies (Dako Corp., Carpenteria, Calif.) used in the dilution provided by the manufacturer to confum the inability of the anti-rabbits IgG antibody to bind non-specifically to the mouse anti-CD34 IgG (see FIG. 8F).

The antibodies and reagents were diluted in Antibody Diluent (Dako Corporation, Carpenteria, Calif.). All incubations were performed for 30 minutes except an initial solubilization incubation with 0.03% H₂O₂ for five minutes. Between all steps, the tissue sections were rinsed in wash buffer (50 mM Tris-HCl, 150 mM NaCl, pH 7.6, with 0.05% Tween-20). Development of the binding reaction was carried out by the addition of 3,3′-diaminobenzidene hydrochloride chromogens provided by the manufacturer in a kit to Buffered Substrate solution (included in kit) for five minutes, followed by a final wash in wash buffer. The tissue sections were counterstained with hematoxylin (Sigma, St. Louis, Mo.), dehydrated in ethanol, and cleared with xylene.

The results were visualized by Olympus BX40 light microscopy (ACCU-SCOPE Instrument Co., NY). Images were taken using Olympus IX50 microscopy and modified with Microsoft PowerPoint 98.

The results indicated that tropomyosin protein is widely expressed in prostate tissue, whereas E-cadherin is expressed by epithelial cells only and CD34 is expressed by endothelial cells only. The results also indicated that the polymer supernatant was able to specifically target the cells expressing each of these proteins by binding to primary mouse IgG. Binding of the polymer supernatant and HRP labeled polymer, as conjugated to goat anti-rabbit IgG, was specific, and the lactoperoxidase itself was not detectable.

### Example 2

### Confirmation of the Functionality of a Conjugated Lactoperoxidase Enzyme

This example provides representative methods for confirming the functionality of a lactoperoxidase enzyme in a DCTA molecule (“polymer”) in vitro.

Bovine serum albumin protein (20 μg) was incubated with either lactoperoxidase (100 ng; lane 2), polymer pellet (lane 4, 5 μl), or polymer supernatant (lane 6, 5 μl) in 0.1 M sodium phosphate buffer, pH 6.25. A 0.4 mCi aliquot of ¹²⁵I was added and the reactions were initiated by the addition of H₂O₂ (10 μg/ml). After a 10-minute incubation, the labeling reaction was halted by the addition of a final concentration of 0.15% NaN₃. The protein mixture was precipitated by the addition of cold acetone followed by a two hour incubation at −70° C. The samples were centrifuged for 15 minutes at 6,500×g. The supernatant was discarded and the protein pellets were lysed in 2× Tris-glycine-SDS sample buffer+10% β-mercaptoethanol, and resolved on a 4-20% SDS-PAGE gel. The gel was placed in a heat-sealed pouch and exposed to X-ray film ¹²⁵I incorporation into the proteins was measured using a gamma counter, COBRA Auto-Gamma (Packard Instrument Co, Downer Grove, Ill.). The following readings were obtained: 665244 CPM/μl for the sample containing lactoperoxidase only; 4124588 CPM/μl for the sample containing lactoperoxidase and BSA, 1090196 CPM/μl for the sample containing polymer pellet only; 2142554 CPM/μl for the containing polymer supernatant only, and 5315076 CPM/μl for the sample containing polymer supernatant and BSA.

These results (as shown in FIG. 4) indicate that lactoperoxidase in both polymer supernatant and polymer pellet samples retains its functional ability to label a single protein with ¹²⁵I. The gels also illustrate that more enzymatic activity in present in the samples containing polymer supernatant than those containing polymer pellet.

### Example 3

### Confirmation of the Ability of a Polymer-Conjugated Lactoperoxidase Enzyme to ¹²⁵-Label Proteins

This example provides methods for confirming the functionality of a lactoperoxidase enzyme in a DCTA molecule (“polymer”) in a human tissue sample containing multiple proteins.

A prostate tissue section was lysed in 200 μl of an extraction buffer composed of equal volumes of 2× Tris-glycine-SDS sample buffer and T-PER, in addition to 10% β-mercaptoethanol. The lysate was incubated at 70° C. for two hours, then centrifuged at 14,000×g for 10 minutes, and the supernatant was removed and used for further analysis. Lysates of prostate tissue (5 μl) were incubated with lactoperoxidase (100 ng), polymer pellet (lane 2, 5 μl) or polymer supernatant (lane 4, 5 μl). Polymer pellet (lane 3, 5 μl), and polymer supernatant (lane 5, 5 μl) were also incubated in the absence of lysates. All reactions were performed in 0.1 M sodium phosphate buffer, pH 6.25.

A 1 mCi aliquot of ¹²⁵I was added and the reactions were initiated by the addition of H₂O₂ (10 μg/ml). After a 10-minute incubation, the labeling reaction was halted by the addition of 0.1% NaN₃, and the protein mixture precipitated by the addition of cold acetone followed by an overnight incubation at −70° C. The samples were centrifuged for 15 minutes at 6,500×g. The supernatant was discarded and the protein pellets were lysed in 2× Tris-glycine-SDS sample buffer and resolved by 4-20% SDS-PAGE. The gels were placed in a heat-sealed and exposed to X-ray film ¹²⁵I incorporation into the proteins was measured using the COBRA Auto-Gamma counter. The following readings were obtained: 1.3×10⁶ CPM/μl for the sample containing lactoperoxidase+lysates; 0.92×10⁶ CPM/μl for the sample containing polymer pellet+lysates; 0.94×10⁶ CPM/μl for the sample containing polymer pellet−lysates; 5.0×10⁶ CPM/μl for the sample containing polymer supernatant+lysates, and 1.5×10⁶ CPM/μl for the sample containing polymer supernatant−lysates.

These results (as shown in FIG. 5) indicate that lactoperoxidase, as present in the DCTA molecule in both polymer supernatant and polymer pellet samples, retains its functional activity. Lactoperoxidase in both samples is able to label multiple proteins in a mixture with ¹²⁵I. Furthermore, the polymer supernatant sample contains more active lactoperoxidase than the polymer pellet sample.

### Example 4

### Confirmation of the Ability of a DCTA Molecule to ¹²⁵I-Label Proteins Embedded in a Tissue Section

This example provides representative methods for confirming that a DCTA molecule (“polymer”) is able to selectively label proteins embedded in a tissue section.

Frozen prostate tissue sections placed on glass slides were thawed, dehydrated and hydrated. The tissue sections were equilibrated in 0.1 M sodium phosphate buffer, pH 6.25. H₂O₂ (10 μg/ml), ¹²⁵I (1 mCi), and either lactoperoxidase (lane 1, 100 ng), polymer pellet (lane 2, 5 μl), or polymer supernatant (lane 3, 5 μl) were added in microfuge tubes, mixed gently, and then added directly on top of the tissue sections. The sections were covered with a cover slip and incubated for 10 minutes. The tissue sections were rinsed in PBS and incubated in 0.1% NaN₃ for at least 5 minutes. The sections were dehydrated, allowed to dry, and lysed in an extraction buffer composed of equal volumes of 2× Tris-glycine-SDS sample buffer and Tissue Protein Extraction Reagent (T-PER, Pierce, Rockford, Ill.), in addition to 10% P-mercaptoethanol. The lysed tissue was transferred into microfuge tubes and incubated at 70° C. for 2 hours. The samples were centrifuged for 10 minutes at 14,000×g, and the supernatant was removed and used for further analysis. 20 μl of the supernatant was resolved on 4-20% SDS-PAGE (InVitrogen, Carlsbad, Calif.). The gel was heat-sealed in a pouch and exposed to X-ray film ¹²⁵I incorporation into the proteins was measured using the COBRA Auto-Gamma counter (Packard, Clearwater, Minn.). The following readings were obtained: 43313 CPM/μl for lactoperoxidase; 16847 CPM/μl for polymer pellet; and 52428 CPM/μl for polymer supernatant.

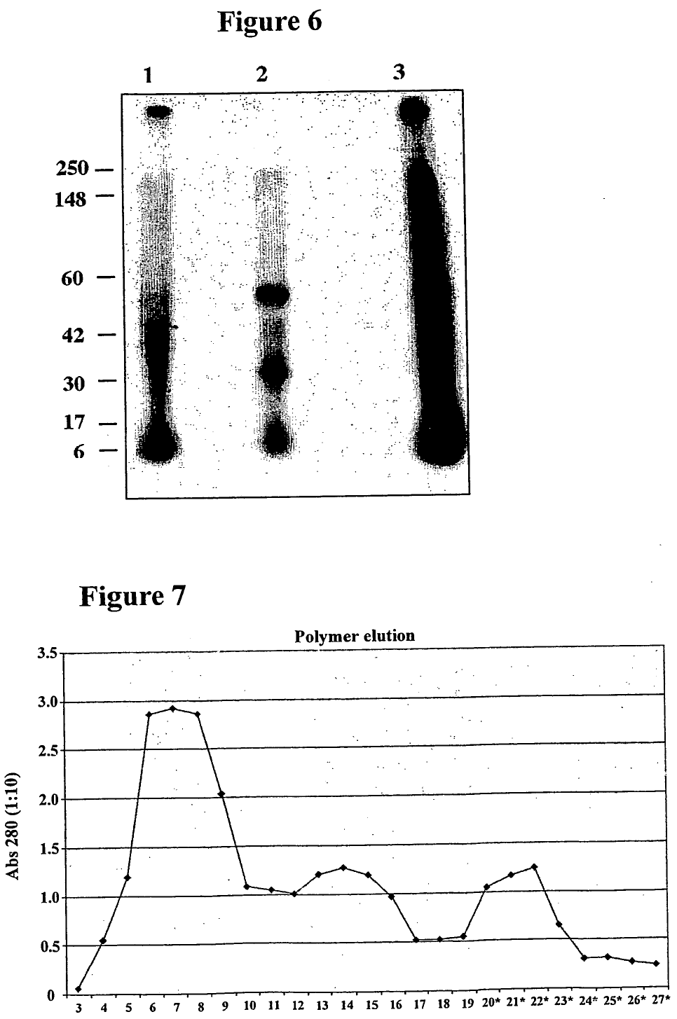

These results (as shown in FIG. 6) indicate that lactoperoxidase in both polymer supernatant and polymer pellet samples, retains its functional ability to label multiple proteins with ¹²⁵I. It is apparent that more active enzyme is present in polymer supernatant than polymer pellet This result may be due to the presence of more active lactoperoxidase in polymer supernatant than polymer pellet and/or the ability of the polymer complex in polymer supernatant to better interact with the tissue as compared to that of polymer pellet.

### Example 5

### Confirmation of the Functionality of a Conjugated Lactoperoxidase Enzyme in the Presence of a Control Reaction

This example provides representative methods for confirming that the labeling by a DCTA molecule (“polymer”) that is seen in a reaction is due to the activity of the DCTA molecule.

Using a DCTA molecule containing lactoperoxidase as the active moiety, synthesized as described above, labeling with ¹²⁵I is performed and measured as set forth in Example 2. Additionally, ¹²⁵I labeling is performed in the absence of the substrate albumin, which serves as a control for measuring background labeling of lactoperoxidase and polymer in the presence of ¹²⁵I (i.e., during analysis, the rates of ¹²⁵I incorporation into the controls are subtracted from the experimental samples, to allow comparison of the rate of ¹²⁵I incorporation by the DCTA molecule with the polymer).

This disclosure provides methods for directly targeting and analyzing cells or distinguishing components of interest from complex, heterogeneous tissue. The disclosed methods allow the targeted cells or cellular components to be procured for subsequent analysis or directly analyzed without the need for physical separation of the targeted cells from other cells or molecules in the population. It will be apparent that the precise details of the methods described may be varied or modified without departing from the spirit of the described invention. We claim all such modifications and variations that fall within the scope and spirit of the claims below.

1. A method of analyzing a tissue sample, comprising:
contacting a Direct Cell Target Analysis (DCTA) molecule with the tissue sample under conditions that allow at least a portion of the DCTA molecule to interact with at least a portion of the tissue sample, wherein the DCTA molecule comprises a targeting moiety, capable of localizing the DCTA to target cells or components within the sample; and an active moiety, capable of generating a detectable signal or product;
activating the active moiety of the DCTA molecule; and
detecting the signal or product generated by the activated second moiety, thereby analyzing the tissue sample.

2. The method of claim 1, wherein the tissue sample comprises biopsy material, a tissue section, a cell culture preparation, a cytology preparation, cells in vitro, or cells in vivo.

3. The method of claim 1, wherein the targeting moiety comprises a variable region of an antibody binding domain.

4. The method of claim 1, wherein the targeting moiety is a generalized targeting moiety and comprises a variable region of a secondary antibody binding domain.

5. The method of claim 1, wherein the targeting moiety comprises a ligand that specifically binds to a receptor protein within or upon target cells in the tissue sample.

6. The method of claim 1, wherein the targeting moiety comprises a nucleic acid molecule capable of hybridizing to a complementary sequence within the target tissue.

7. The method of claim 1, wherein the active moiety comprises: (1) a reverse transcriptase molecule and the detectable products are cDNA transcripts; or (2) a DNA polymerase molecule and the detectable products are DNA transcripts.

8. (canceled)

9. The method of claim 7, wherein one or more components that are necessary for generation of the detectable products are externally provided.

10. The method of claim 9, wherein at least one of the provided components is a labeled nucleotide.

11. The method of claim 10, wherein the labeled nucleotide is labeled with an isotope or a fluorophore.

12. The method of claim 1, wherein the active moiety comprises a lactoperoxidase molecule and the detectable products comprise iodinated tryptophan or tyrosine residues.

13. The method of claim 1, wherein the active moiety comprises lactoperoxidase and the detectable products comprise ¹²⁵I labeled proteins.

14. The method of claim 1, wherein the detectable signal is visualized without physical separation of the analyzed products from the sample.

15. The method of claim 1, wherein the detectable products are separated from the sample prior to analysis.

16. The method of claim 1, further comprising quantifying the detectable products.

17. The method of claim 1, wherein the detectable products are amplified during analysis.

18. The method of claim 1, wherein the DCTA molecule comprises at least one targeting moiety and at least one active moiety each covalently linked to a polymer linker.

19. The method of claim 1, wherein the DCTA molecule comprises a poly(1-lysine hydrobromide) polymer conjugated to lactoperoxidase and goat anti-mouse IgG antibody.

20. A method for screening for a disease in a subject, comprising using the method of claim 1 to analyze a tissue sample from the subject for the presence of a protein, or a nucleic acid encoding the protein, wherein the presence of the protein or the nucleic acid encoding the protein in sample from the subject is indicative of the disease in the subject.

21. A method for screening for a disease in a subject, comprising using the method of claim 1 to compare expression levels of a nucleic acid in a tissue sample from the subject, wherein elevated or decreased expression levels of the nucleic acid compared to a sample from a control subject known not to have the disease is indicative of the disease in the subject.

22. A method for screening for a disease in a subject, comprising using the method of claim 1 to screen for a nucleic acid in a sample from the subject, wherein absence of the nucleic acid in the target cells is a biochemical marker of disease in the subject.

23. A method for screening for a disease in a subject, comprising using the method of claim 1 to screen for a hormone in a sample from the subject, wherein the absence of the hormone in the target cells is a biochemical marker of disease in the subject.

24. The method of claim 1, wherein the method is a method for screening for a disease in a subject, comprising using the method of claim 1 to screen for the presence of a mutation in the nucleic acid in a sample from the subject, wherein the presence of such a mutation is a genetic marker of disease in the subject.

25. The method of claim 22, wherein the disease comprises a neoplasia.

26. The method of claim 1, wherein the DCTA is automated.

27-33. (canceled)

34. The method of claim 23, wherein the disease comprises a neoplasia.

35. The method of claim 24, wherein the disease comprises a neoplasia.
